(19) United States
(12) Patent Application Publication
(10) Pub. No.: US 2024/0197466 A1
(43) Pub. Date: Jun. 20, 2024

(54) MULTI-COMPONENT BREAST IMPLANT

(71) Applicant: Tepha, Inc., Lexington, MA (US)

(72) Inventors: Skander Limem, Lynnfield, MA (US); Kemal Sariibrahimoglu, Sunnyvale, CA (US); Timothy John Butler, Pelham, NH (US); Simon F. Williams, Cambridge, MA (US)

(73) Assignee: Tepha, Inc., Lexington, MA (US)

(21) Appl. No.: 18/287,084

(22) PCT Filed: Apr. 15, 2022
(86) PCT No.: PCT/US2022/024954

§ 371 Date: Oct. 16, 2023

Related U.S. Application Data
(60) Provisional application No. 63/176,450, filed on Apr. 19, 2021.

(51) Int. Cl. A61F 2/12; A61L 27/52; A61L 27/56; A61L 27/58; B33Y 10/00

(52) U.S. Cl. CPC A61F 2/12; A61L 27/52; A61L 27/56; A61L 27/58; B33Y 10/00; A61F 2210/0004; A61L 2430/04

(57) ABSTRACT

Absorbable implants can be used to create volume and shape in the breast of a patient with regenerated tissue. The implants are transient comprising a reinforced matrix comprising a load bearing absorbable macroporous network with an open pore structure, at least partially filled with one or more degradable hydrogels or water-soluble polymers that degrade progressively, from the surface of the implant towards the core of the implant, and direct tissue ingrowth in the same direction. The hydrogels and water-soluble polymers help prevent fluid accumulation in the implant prior to tissue ingrowth. The macroporous networks are absorbed after tissue ingrowth has occurred, and a load bearing support is no longer necessary. The implants are particularly suitable for use in plastic surgery procedures, for example, to regenerate or augment breast tissue following mastectomy or in mastopexy procedures, and can provide an alternative to the use of permanent breast implants in these procedures.

### FIELD OF THE INVENTION

The present invention generally relates to three-dimensional implants with a load bearing reinforced macroporous network at least partly filled with one or more hydrogels, one or more water-soluble polymers, or combinations thereof, suitable for replacing breast tissue. More particularly, the implants are preferably absorbable, and comprise one or more hydrogels, water-soluble polymers, or combinations thereof, that degrade to permit tissue in-growth and minimize the accumulation of fluid during tissue in-growth. The implants are designed to replace or increase the volume of soft tissue when implanted in the breast.

### BACKGROUND OF THE INVENTION

Breast reconstruction following mastectomy has become an integral and important part of breast cancer treatment with the surgery providing the patient with both aesthetic and psychosocial benefits. In the US, nearly 65% of breast reconstruction procedures now use a tissue expander to create a pocket for a permanent breast implant in the first step of the procedure. In some patients, a pocket for the breast implant can be formed without the use of a tissue expander. Once a pocket has been created, the tissue expander is removed, and replaced with a permanent breast implant in a second step.

Breast implants can also be used in breast augmentation and mastopexy procedures to augment breast size. In the latter procedure, a breast lift is combined with breast augmentation. Most commonly, the breast implant is placed in a pocket under the breast tissue, but in some cases, it is implanted under the chest wall.

Breast implants differ in dimensions, shape, and surface texture. A wide variety of different dimensions are available allowing the surgeon and patient to select from a range of projections, heights, widths and overall volume. In terms of shape, there are round and anatomically shaped implants, and the surfaces of the implants may be smooth, micro-textured or macro-textured. Generally, round implants have smooth surfaces, whereas anatomically shaped implants have dimpled micro- or macro-textured surfaces.

A growing number of patients considering breast reconstruction and augmentation are however reluctant to have permanent breast implants placed in their breasts. This is particularly the case for women that have had a mastectomy, and are now considering breast reconstruction. Some of these patients do not want to have a permanent foreign body placed in their breasts, and they don't want to run the risk of complications that can develop with permanent breast implants. The complications include a risk of: capsular contraction requiring reoperation, rupture or deflation of the implant, development of anaplastic large cell lymphoma (ALCL), infection, and movement of the implants causing asymmetry of the breasts.

WO2016/038083 to Hutmacher discloses an implant for tissue reconstruction which comprises a scaffold structure that includes a void system for the generation of prevascularized connective tissue with void spaces for cell and/or tissue transplantation. See Abstract Hutmacher.

US2018/0206978 to Rehnke discloses an internal brassiere device made from a pleated scaffold that can be used in breast augmentation patients.

WO2018/078489 to Danze discloses a device to be implanted in a subject's body to form an implant for replacing and/or increasing a volume of soft tissue, the device being of the type including a three-dimensional frame which defines an inner space in said frame. The frame is typically bio-absorbable and includes two side apertures forming a transverse passage for inserting a vascular pedicle; the device further comprises at least two bio-absorbable textile sheets that can be stacked on each other in the inner space of said frame. See Abstract Danze.

US2020/0375726 to Limem discloses implants formed from unit cells suitable for use in breast reconstruction.

WO2019/217335 to Toro Estrella discloses bio-scaffold structures comprising a plurality of connected unit cells, wherein each unit cell includes at least one opening connected to an internal volume.

WO2004/052768 to Morrison discloses a method of producing vascularized tissue utilizing a vascular pedicle enclosed in a chamber and implanted in a donor. A vascularized tissue graft suitable for transplantation, and a method of repairing a tissue deficit using a vascularized tissue graft.

WO2019/043950 to Chhaya discloses implants for soft tissue reconstruction comprising a plurality of unit cells arranged to form a reversibly compressible porous three-dimensional lattice structure with a bulk porosity of at least 50%.

Zhou et al. “Tuning the mechanics of 3D-printed scaffolds by crystal lattice-like structural design for breast tissue engineering”, Biofabrication, 12 (2020) 015023 discloses additive manufactured breast scaffolds prepared using polyurethane.

Notwithstanding the above, there is still a need for improved breast implants that, when implanted, can generate new breast tissue with a specific and desirable appearance.

### SUMMARY OF THE INVENTION

Breast implants described herein assist the surgeon in reconstructing the breast particularly following mastectomy, enhancing the appearance of the breast, augmenting the size of the breast, reconstructing lost or missing breast tissue, enhancing the tissue structure of the breast, increasing the soft tissue volume of the breast, restoring the natural feeling of soft tissue in the breast, and delivering biological and synthetic materials to assist in tissue regeneration, repair, and reconstruction of the breast.

The breast implants are configured with a surface, a core, a back area, and a front area opposite the back area. The back area of the implant is designed to be placed in the breast of a patient on or near the chest wall. The front area comprises a front bottom for placement in the lower pole of the breast, a front top for placement in the upper pole of the breast, and a front intermediate-region for placement under the skin of the patient. In embodiments, the breast implant has a longitudinal axis defined by the axis between the back area and front area of the implant.

In embodiments, the breast implant comprises a reinforced matrix. In embodiments, the reinforced matrix comprises a load bearing macroporous network with an open cell structure at least partly filled with one or more degradable hydrogels, water-soluble polymers, or combinations thereof. In embodiments, the load bearing macroporous network with an open cell structure has a compressive strength of at least 0.1 kgf at 30% strain. In embodiments, the load bearing macroporous network with an open cell structure has a compressive modulus of 0.1 kPa to 10 MPa at 5 to 15% strain. In embodiments, the load bearing macroporous network with an open cell structure has a loss modulus of 0.3 to 100 kPa. In embodiments, the load bearing macroporous network with an open cell structure comprises fibers, filaments, 3D print lines, or struts. In embodiments, the load bearing macroporous network is absorbable. In embodiments, the load bearing macroporous network has a predictable rate of degradation, and a predictable strength retention in vivo. In embodiments, the reinforced matrix comprises a degradable load bearing macroporous network. In embodiments, the one or more degradable hydrogels, water-soluble polymers, or combinations thereof, of the implant degrade before the absorbable load bearing macroporous network.

In embodiments, the breast implants provide a transient scaffold for tissue ingrowth. Following implantation, the implant is designed to be progressively invaded by connective tissue and bloods vessels, and become well integrated in the breast. The breast implants minimize the accumulation of fluid following implantation, which can occur when implants with large void volumes are implanted in the breast. Upon implantation, one or more degradable hydrogels, water-soluble polymers, or combinations thereof, that fill at least part of the implant gradually degrade permitting tissue ingrowth without significant accumulation of fluid in void spaces of the implant. Accumulation of fluid in void spaces is minimized by the presence of the one or more hydrogels, water-soluble polymers, or combinations thereof, prior to tissue ingrowth. Preferably, the hydrogel, water-soluble polymer, or combination thereof, structure of the implant degrades progressively from the surface of the implant towards the core of the implant, with tissue ingrowth proceeding in the same direction from the surface of the implant to the core. Degradation of the hydrogel, water-soluble polymer, or combination thereof, preferably precedes the degradation of the load bearing macroporous network allowing the load bearing macroporous network to provide a scaffold for tissue ingrowth and structural support once the hydrogel, water-soluble polymer, or combination thereof, degrades.

In embodiments, the load bearing macroporous network of the implant retains strength long enough to allow the shape of the breast at the implant site to be transitioned from the implant to new tissue. The implant needs to maintain its shape for a prolonged period in order to direct re-modeling of the patient's tissue. The implant preferably provides support of the breast until support is transitioned from the implant to new tissue. Preferably, no loss of support, or minimal loss of support, for the shape of the breast occurs during this transition period. The shape of the breast implant is maintained for a prolonged period in order to direct tissue in-growth into the implant, and produce the desired breast shape.

In embodiments, the breast implants comprise two or more degradable polymers selected from hydrogels and water-soluble polymers preferably with different rates of degradation. In an embodiment, the breast implant comprises at least one of a first hydrogel or a first water-soluble polymer and at least one of a second hydrogel or a second water-soluble polymer, and the second hydrogel or second water-soluble polymer surrounds the first hydrogel or the first water-soluble polymer. The degradation rate of the second hydrogel or second water-soluble polymer is preferably faster than the degradation rate of the first hydrogel or the first water-soluble polymer. Degradation of the second hydrogel or the second water-soluble polymer exposes a portion of the load bearing macroporous network allowing tissue in-growth into the volume of the implant that was occupied by the second hydrogel or the second water-soluble polymer. Meanwhile, accumulation of fluid in the volume occupied by the first hydrogel or the first water-soluble polymer is prevented by the continuing presence of the first hydrogel or the irst water-soluble polymer in the implant. In embodiments, the first hydrogel or the first water-soluble polymer degrades after degradation of the second hydrogel or the second water-soluble polymer, and allows tissue ingrowth into the volume occupied by the first hydrogel or the first water-soluble polymer. Complete degradation of both hydrogels and/or water-soluble polymers allows tissue ingrowth into the entire load bearing macroporous network, and the formation of new breast tissue throughout the implant. In embodiments, the load bearing macroporous network is transient, and is completely degraded after tissue ingrowth and structural support is no longer required.

In embodiments, the breast implant comprises a load bearing macroporous network, a first water-soluble polymer, and a second water-soluble polymer, wherein the second water-soluble polymer surrounds the first water soluble polymer. The degradation rate of the second water-soluble polymer is preferably faster than the degradation rate of the first water-soluble polymer.

In embodiments, the breast implant comprises a load bearing macroporous network, a hydrogel, and a water-soluble polymer. In embodiments, the water-soluble polymer degrades faster than the hydrogel degrades, and the water-soluble polymer surrounds the hydrogel. In embodiments, the hydrogel degrades faster than the water-soluble polymer degrades and the hydrogel surrounds the water-soluble polymer.

In embodiments, the breast implant comprises a load bearing macroporous network and three hydrogels. Preferably, the three hydrogels have three different degradation rates. In embodiments, the breast implant comprises a first, a second, and a third hydrogel with the third hydrogel degrading the fastest, followed by the second hydrogel, with the first hydrogel being the last to degrade. The breast implant is formed with the first hydrogel located at the core of the breast implant surrounded by the second hydrogel that will degrade faster than the first hydrogel. The second hydrogel is surrounded by the third hydrogel. The third hydrogel degrades faster than the second hydrogel. Together, the hydrogels create a gradient of degradation rates with degradation occurring progressively slower moving in the direction from the surface of the implant to its core. Preferably, the load bearing macroporous network degrades after the three hydrogels have degraded. In embodiments, the one or more hydrogels in the implant degrade in less than 3 months, and the load bearing macroporous network degrades in 3-24 months. In embodiments, the load bearing macroporous network has a strength retention of 50% at 3 months. In embodiments, the implant comprises three hydrogels, and the third (outermost) hydrogel degrades within one month, the second hydrogel degrades within two months, and the first (innermost) hydrogel degrades within three months. In embodiments, the implant comprises three hydrogels, and the third (outermost) hydrogel degrades within one month, the second hydrogel degrades within three months, and the first (innermost) hydrogel degrades within six months. In embodiments, the load bearing macroporous network has a strength retention of 50% at 6 months.

In embodiments, the breast implant comprises a macroporous load bearing network, and three water-soluble polymers. Preferably, the three water-soluble polymers have different rates of degradation. In embodiments, the breast implant comprises a first, a second, and a third water-soluble polymer with the third water-soluble polymer degrading the fastest, followed by the second water-soluble polymer, with the first water-soluble polymer being the last to degrade. The breast implant is formed with the first water-soluble polymer located at the core of the breast implant surrounded by the second water-soluble polymer that will degrade faster than the first water-soluble polymer. The second water-soluble polymer is surrounded by the third water-soluble polymer.

In embodiments, the breast implant comprises a macroporous load bearing network, a water-soluble polymer and two hydrogels, or comprises a macroporous load bearing network, two water-soluble polymers and one hydrogel. The slowest degrading water-soluble polymer or hydrogel is located at the core of the breast implant, and is surrounded by a faster degrading second water-soluble polymer or hydrogel. The second water-soluble polymer or hydrogel is surrounded by the fastest degrading water-soluble polymer or hydrogel.

In embodiments, the breast implants comprise a load bearing macroporous network at least partly filled with a first hydrogel and a second hydrogel, or a first water-soluble polymer and a second water-soluble polymer, wherein the second hydrogel or the second water-soluble polymer at least partially surrounds the first hydrogel or the first water-soluble polymer, and the second hydrogel or the second water-soluble polymer degrades faster than the first hydrogel or the first water-soluble polymer. In embodiments, the second hydrogel or the second water-soluble polymer surrounds the first hydrogel or the first water-soluble polymer except in the back area of the implant where the implant is in contact with the chest wall. In embodiments, the hydrogels and the water-soluble polymers degrade before the macroporous network is completely degraded. In embodiments, the breast implants further comprise a third hydrogel or a third water-soluble polymer, wherein the third hydrogel, or the third water-soluble polymer, at least partly surrounds the second hydrogel or the second water-soluble polymer, and the third hydrogel or the third water-soluble polymer degrades faster than the second hydrogel or the second water-soluble polymer. In embodiments, the three hydrogels or the three water-soluble polymers degrade before the macroporous network is completely degraded. In embodiments, the second hydrogel or the second water-soluble polymer surrounds the first hydrogel or the first water-soluble polymer and the third hydrogel or the third water-soluble polymer surrounds the second hydrogel or the second water-soluble polymer except in the back area of the implant where the implant is in contact with the chest wall.

In embodiments, the breast implant comprises a reinforced macroporous network with a plurality of interconnected voids; and a first set of sacrificial void occupiers adapted to temporarily occupy a first set of voids until tissue grows therein. In embodiments, the breast implant further comprises a second set of voids arranged to surround the first set of voids, and a second set of sacrificial void occupiers adapted to temporarily occupy the second set of voids until tissue grows therein, and wherein the second set of void occupiers is absorbable prior to the first set of void occupiers. In embodiments, the first set of sacrificial void occupiers is a first hydrogel or a first water-soluble polymer. In embodiments, the first and the second set of sacrificial void occupiers, and macroporous network are fabricated and arranged together to form the implant by 3D printing. In embodiments, the second set of sacrificial void occupiers is a second hydrogel or a second water-soluble polymer. In embodiments, the second set of void occupiers is absorbed before the first set of void occupiers, and the macroporous network degrades after the first set of void occupiers is degraded. In embodiments, the first set of void occupiers is absorbed within one year of implantation of the implant in the breast.

In embodiments, the implant has a shape and size suitable for use in breast surgery procedures, including breast augmentation, breast reconstruction and mastopexy. In embodiments, the breast implant has a dome-like shape, a round shape, a teardrop shape, or an anatomical shape. In embodiments, the implant has a shape designed to provide the breast with a desirable anatomical shape.

In embodiments, the front bottom of the breast implant has a convex exterior surface. The convex exterior surface is sized and shaped to enhance the profile of the lower pole of the breast, and preferably approximates the anatomical feature of the lower pole of the breast.

In embodiments, the breast implant comprises an opening for insertion of tissue into the implant. In embodiments, the opening is located on the back area of the implant. In embodiments, the opening is located on the back area of the implant, and has a longitudinal axis between the back and front areas of the implant. In embodiments, the implant may have an opening that is a hollow core defining a longitudinal axis between the back and front areas of the implant.

In embodiments, the implants may comprise two or more openings to allow the insertion of multiple vascular pedicles, or other masses of tissue into the implant.

In embodiments, the implants comprise an external shell enclosing the reinforced matrix.

In embodiments, the implants comprise one or more anchors, fasteners or tabs to fixate the implant to the breast.

In embodiments, the implants are absorbable.

In embodiments, the load bearing macroporous network comprises one or more absorbable polymers. In embodiments, the one or more absorbable polymers comprise, or is prepared from, one or more monomers selected from the group: glycolide, lactide, glycolic acid, lactic acid, 1,4-dioxanone, trimethylene carbonate, 3-hydroxybutyric acid, 3-hydroxybutyrate, 3-hydroxyhexanoate, 3-hydroxyoctanoate, 4-hydroxybutyric acid, 4-hydroxybutyrate, ε-caprolactone, 1,4-butanediol, 1,3-propane diol, ethylene glycol, glutaric acid, malic acid, malonic acid, oxalic acid, succinic aid, and adipic acid, or wherein the polymeric composition comprises poly-4-hydroxybutyrate or copolymer thereof, or poly(butylene succinate) or copolymer thereof.

In embodiments, the absorbable polymers used in the preparation of the implants have weight average molecular weights of 50 to 1,000 kDa, more preferably 90 to 600 kDa, and even more preferably from 200 to 450 kDa relative to polystyrene determined by GPC.

In embodiments, the load bearing macroporous network with an open cell structure comprises fibers, filaments, 3D print lines, or struts with one of the following properties: a tensile strength higher than 25 MPa, a tensile modulus less than 300 MPa, an elongation at break greater than 100%, a melting temperature of 60° C. or higher, a glass transition temperature of less than 0° C., an average diameter or width of 10 μm to 5 mm, a breaking load of 0.1 to 200 N, and an elastic modulus of 0.05 to 1,000 MPa.

In embodiments, the implants comprise hydrogels that are degradable, and preferably two or more hydrogels with different rates of degradation. In embodiments, the hydrogels are homopolymeric, copolymeric or multipolymer interpenetrating polymer hydrogels. The hydrogels may be amorphous, semicrystalline or crystalline. The hydrogels may comprise chemical or physical crosslinking. The hydrogels may be photo-crosslinked. The hydrogels may be nonionic, ionic, amphoteric electrolyte containing both acidic and basic groups or zwitterionic containing both anionic and cationic groups. The hydrogels may comprise natural polymers, including proteins and polysaccharides. Examples include collagen, gelatin, fibrin, elastin, starch, cellulose, methylcellulose, carboxymethylcellulose, hydroxypropyl methyl cellulose, hyaluronan, hyaluronic acid, alginate, chitosan, carrageenan, pectin, dextran, β-glucan, gellan, welan, xanthan, and agarose. The hydrogels may comprise synthetic polymers. Examples include polyvinyl alcohol, poly(vinyl methyl ether), polyethylene glycol, polypropylene glycol, polyurethanes, polyphosphazenes, polypeptides, poly(N-isopropyl acrylamide), poly(vinylpyrrolidone), polymethacrylic acid, and polyacrylates and copolymers.

In embodiments, the implants comprise water-soluble polymers that are degradable in vivo. In embodiments, the water-soluble polymers degrade in vivo to allow tissue ingrowth into the load bearing macroporous network. In embodiments, the water-soluble polymers are not hydrogels. The water-soluble polymers may be natural polymers, biosynthetic or synthetic polymers. Examples of water-soluble polymers include polyethylene glycol, polyvinyl pyrrolidone, polyvinyl alcohol, polyacrylic acids, polyacrylamides, polyphosphates, polyoxazolines, divinyl ether-maleic anhydride, N-(2-hydroxypropyl) methacrylamide, and polyphosphazenes.

In embodiments, the breast implants may comprise one or more of the following: autologous fat, fat lipoaspirate, injectable fat, cells, adipose cells, fibroblast cells, stem cells, gels, hydrogels, hyaluronic acid, collagen, water-soluble polymer, antimicrobial, antibiotic, bioactive agent, and diagnostic device. Following implantation, the implant is designed to be invaded by connective tissue and bloods vessels, and become well integrated in the breast. In embodiments, the implant may be an adipose tissue engineering scaffold.

In embodiments, the breast implant has a compressive modulus at 5 to 15% strain of 0.1 kPa to 10 MPa, more preferably 0.3 kPa to 1 MPa, and even more preferably 3 kPa to 200 kPa. The compressive modulus allows the implant to be compressed, and recover from compression. The breast implant is engineered so that the breast does not feel hard after implantation of the implant, but is soft to the touch, and feels like a natural breast. In embodiments, the breast implant allows the surgeon to restore or augment breast mass while maintaining or restoring the tactile sensation of the breast.

In embodiments, the breast implant has a loss modulus of 0.1 kPa to 5 MPa, preferably 0.3 kPa to 1 MPa, and even more preferably 0.3 kPa to 100 kPa.

In embodiments, the breast implant has a compression resilience of 1 to 80%.

In embodiments, the breast implant is adapted to recover post deformation/compression with a minimum 50%, more preferably 75%, and most preferably 90% or more.

In embodiments, the implant can be delivered to the breast in a minimally invasive manner.

In embodiments, the implant is compressible and may be delivered into the breast through a funnel, such as a Keller funnel. In embodiments, the implant may be delivered into the breast through a funnel with a neck diameter (the narrowest part of the funnel) of 1 to 3 inches, and more preferably 1.5 to 2.5 inches. In embodiments, the implant may be delivered through a funnel with a neck diameter of 1 to 3 inches, and recover at least 70% of the implant's volume after delivery through the neck of the funnel into the breast. In embodiments, the implant has a compressive modulus to allow delivery of the implant through a funnel with a neck diameter of 1 to 3 inches, and the implant is able to recover at least 70% of the implant's volume after passage through the funnel's neck.

In embodiments, the implants have an endotoxin content of less than 20 endotoxin units per implant.

In embodiments, the implants are sterilized implants. The implants can be sterilized by a range of techniques including without limitation ethylene oxide, electron beam, or gamma-irradiation.

In embodiments, methods are provided for manufacturing the implants comprising a reinforced matrix, wherein the reinforced matrix comprises a load bearing macroporous network with an open cell structure at least partly filled with one or more degradable hydrogels, one or more water-soluble polymers, or a combination of degradable hydrogels and water-soluble polymers, and wherein the implant comprises a back area for placement on the chest wall of a patient, a front area opposite the back area, the front area comprising a front bottom for placement in the lower pole of the breast, a front top for placement in the upper pole of the breast, and a front intermediate-region for placement under the skin of the patient. The methods provide for the manufacture of a degradable load bearing macroporous network with an open cell structure having a compressive strength of at least 0.1 kgf at 30% strain, a compressive modulus of 0.1 kPa to 10 MPa at 5 to 15% strain, or a loss modulus of 0.3 to 100 kPa. In embodiments, the load bearing macroporous network with an open cell structure is manufactured by forming a network of filaments by 3D printing a polymeric composition. In embodiments, the load bearing macroporous network is formed by extrusion-based additive manufacturing, selective laser melting, fused deposition modeling, fused filament fabrication, melt extrusion deposition, printing of a polymer slurry or solution using a coagulation bath, and printing using a binding solution and granules of polymer powder. In embodiments, the infill density of 3D printed filaments in the load bearing macroporous network is between 1% and 60%, and more preferably between 5% and 25%.

In embodiments, the load bearing macroporous network with an open cell structure is manufactured by 3D printing of a polymeric composition, wherein the polymeric composition comprises a polymer selected from a polymer or copolymer comprising, or prepared from, one or more of the following monomers: glycolide, lactide, glycolic acid, lactic acid, 1,4-dioxanone, trimethylene carbonate, 3-hydroxybutyric acid, 3-hydroxybutyrate, 4-hydroxybutyric acid, 4-hydroxybutyrate, ε-caprolactone, 1,4-butanediol, 1,3-propane diol, ethylene glycol, glutaric acid, malic acid, malonic acid, oxalic acid, succinic aid, and adipic acid, or wherein the polymeric composition comprises poly-4-hydroxybutyrate or copolymer thereof, or poly(butylene succinate) or copolymer thereof.

In embodiments, the method of manufacturing the load bearing macroporous network of the implant comprises forming, preferably by 3D printing, the filaments of the network from a polymer with one or more of the following properties: (i) an elongation at break greater than 100%; (ii) an elongation at break greater than 200%; (iii) a melting temperature of 60° C. or higher, (iv) a melting temperature higher than 100° C., (v) a glass transition temperature of less than 0° C., (vi) a glass transition temperature between −55° C. and 0° C., (vii) a tensile modulus less than 300 MPa, and (viii) a tensile strength higher than 25 MPa. In preferred embodiments, the loading bearing macroporous network of the implant is made from P4HB, PBS, P4HB copolymers or PBS copolymers, by 3D printing.

In embodiments, a 3D printer is used to manufacture the implants, wherein one print head of the 3D printer is used to print the load bearing macroporous network with an open cell structure, and one or more print heads are used to print one or more hydrogels, or one or more water-soluble polymers, so that an implant comprising a macroporous network with an open cell structure at least partly filled with one or more hydrogels and/or one or more water-soluble polymers, is formed. In this manner, an implant may be manufactured wherein the degradation rate of each hydrogel, or degradation rate of each water-soluble polymer, incorporated into the implant decreases moving from the surface of the implant to the core of the implant.

In embodiments, the load bearing macroporous network with an open cell structure at least partly filled with one or more degradable hydrogels, one or more degradable water-soluble polymers, or combinations thereof, is manufactured with a 3D printer with two or more print heads. In embodiments, the implant is formed with a 3D printer with two print heads, wherein one print head is used to form the load bearing macroporous network with an open cell structure, for example, using extrusion-based additive manufacturing, for example, fused deposition modeling or fused pellet deposition, and a second print head is used to print a hydrogel or water-soluble polymer, for example, using solution-based 3D printing, so that the hydrogel or water-soluble polymer at least partly fills the load bearing macroporous network. In embodiments, the implant is formed with a 3D printer with at least three print heads, wherein one print head is used to form the load bearing macroporous network with an open cell structure, for example, using fused deposition modeling or fused pellet deposition, a second print head is used to print a first hydrogel, or a first water-soluble polymer, for example, using solution-based 3D printing, so that it fills the core of the load bearing macroporous network, and a third print head is used to print a second hydrogel, or a second water-soluble polymer, for example, using solution-based 3D printing, so that the second hydrogel, or the second water-soluble polymer, surrounds the first hydrogel or the first water-soluble polymer. The second hydrogel has a faster rate of degradation than the first hydrogel. The second water-soluble polymer has a faster degradation rate than the first water-soluble polymer.

In embodiments, methods of manufacturing the implants comprise 3D printing the implant, or 3D printing the loading bearing macroporous network, and adding one or more of the following components: autologous fat, fat lipoaspirate, injectable fat, adipose cells, fibroblast cells, stem cells, gel, hydrogel, hyaluronic acid, collagen, antimicrobial, antibiotic, bioactive agent, and diagnostic device. In embodiments, these components are added to the implant by coating, spraying, immersion or injection.

In embodiments, methods of manufacturing the load bearing macroporous network of the implants comprise particle leaching, phase separation, foaming, lamination and perforation. In embodiments, the methods of manufacturing the load bearing macroporous network of the implants comprise textile processing, including weaving, knitting, braiding, and forming nonwovens, for example, by melt blowing, electrospinning, staple fiber processing, solution spinning, centrifugal spinning, spunbonding, and dry spinning.

In embodiments, the load bearing macroporous network may be a 3D textile, a 3D braided fabric, or a 3D composite.

In embodiments, the implant has a pre-determined three-dimensional shape that can be implanted subcutaneously, between the skin and the breast mound or chest wall of the breast. The breast implant may be implanted in the sub-glandular, sub-pectoral, or subfascial positions. The implant design allows the surgeon to easily control the volumetric ratios of the upper and lower poles of the breast, the extent of protrusion of the breast from the chest wall, and the curvatures of the upper and lower poles of the breast.

In embodiments, the implant serves to provide the surgeon with a means to deliver cells, stem cells, differentiated cells, fat cells, muscle cells, platelets, tissue, pedicles, vascular pedicles, tissue masses, lipoaspirate, extracellular adipose matrix proteins, gels, hydrogels, hyaluronic acid, collagen, bioactive agents, drugs, antibiotics, and other materials to the implant site. Preferably, the cells and tissues delivered by the implants, or coated or injected into the implants, are autologous. The implants may be used for autologous fat transfer. The implants may comprise bioactive agents to stimulate cell ingrowth, including growth factors, cell adhesion factors, cellular differentiating factors, cellular recruiting factors, cell receptors, cell-binding factors, cell signaling molecules, such as cytokines, and molecules to promote cell migration, cell division, cell proliferation and extracellular matrix deposition.

In embodiments, the implants can be implanted to replace and or increase a soft tissue volume or a tissue mass. In embodiments, the implants may further comprise a growth chamber for cells and tissues.

In embodiments, methods are provided for implanting the implants in the breast of a patient. In embodiments, the methods of implantation of the implants comprise: (i) making at least one incision to gain access to the breast tissue of the patient, (ii) separating the skin and subcutaneous fascia from the breast mound of the breast, (iii) positioning the implant sub-glandular, sub-pectoral, or subfascial (iv) securing the implant to nearby tissue, and (v) closing the incisions in the breast. In embodiments, the method of implanting the implants in the breast further comprise coating on the implant, or adding to the implant, one or more of the following components on one or more occasions either prior to implanting the implant in the breast or after implanting the implant in the breast: autologous fat, fat lipoaspirate, injectable fat, adipose cells, fibroblast cells, stem cells, gel, hydrogel, hyaluronic acid or derivative thereof, collagen, antimicrobial, antibiotic, and a bioactive agent. In embodiments, the components are added to the implant by injection, spraying, immersion or coating, but preferably by injection of the components onto or into the macroporous network of the implant. In embodiments, the implant is coated with autologous tissue from the patient prior to implantation, during implantation, or after implantation, or any combination thereof. In embodiments, the method of implantation comprises implanting an implant with an opening sized for insertion of tissue into the implant, and inserting tissue or pedicle, preferably a vascular pedicle, more preferably a vascular pedicle perforator, and even more preferably a pedicle from the small pectoral muscle preferably with a perforator, into the opening of the implant during implantation of the implant. In embodiments, the method of implantation comprises dissecting a pedicle from the patient's small pectoral muscle, preferably with a perforator, and inserting the pedicle in an opening in the implant that is sized to receive the pedicle. In embodiments, the surgeon may insert a pedicle or other tissue mass in the implant prior to, or after, implantation of the implant in a patient. The breast implant can be used in patients that have: (i) undergone mastectomy, (ii) undergone breast lift and have need of an augmentation, (iii) undergone breast reduction and need support and lift of the reduced breast, (iv) undergone prior silicone or saline breast implant breast surgery, and desire that the silicone or saline implant is removed and that there is subsequent reconstruction of the breast to produce a youthful appearance but with a fuller breast and larger size. The implant may also be used in patients that want the feeling of natural breast tissue restored to the breast after removal of their breast tissue. The implant can be used to increase projection of the breast from the chest, and in combination with fat grafting to add volume to the breast.

These advantages as well as other objects and advantages of the present invention will become apparent from the detailed description to follow, together with the accompanying drawings.

### BRIEF DESCRIPTION OF THE DRAWINGS

FIG. 1A is a cross sectional view taken along line A-A of a multi-component breast implant 1 shown in FIG. 1C in accordance with one embodiment of the invention. The breast implant 1 is shown with a surface 2, a core 3, a back area 4 for placement on or near the chest wall of the patient, a front area 5 opposite the back area, a front bottom 6 for placement in the lower pole of the breast, a front top 7 for placement in the upper pole of the breast, a front intermediate-region 8 for placement under the skin of the patient, and a load bearing macroporous network 9 with an open cell structure. The locations within the breast implant of a first hydrogel 10, a second hydrogel 11, and a third hydrogel 12 are also shown.

FIG. 1B is an isometric view of the multicomponent breast implant 1 shown in FIG. 1A with a back area 4 for placement on or near the chest wall of the patient, a front area 5 opposite the back area, a front bottom 6 for placement in the lower pole of the breast, a front top 7 for placement in the upper pole of the breast, a front intermediate-region 8 for placement under the skin of the patient, and the locations within the implant of a first hydrogel 10, a second hydrogel 11, and a third hydrogel 12.

FIG. 1C is a top view of a cross section along the mid plane of breast implant 1 in accordance with one embodiment of the invention. The breast implant is shown with a first hydrogel 10, a second hydrogel 11, and a third hydrogel 12.

FIG. 1D is a second isometric view of a multicomponent breast implant 1 shown in FIG. 1A with a back area 4 and a front area 5.

FIG. 1E is another cross sectional view of the multi-component breast implant 1 shown FIG. 1C taken along A-A with the first, second and third hydrogels (10, 11, 12) removed.

FIG. 1F is an enlarged view of a portion of the load bearing macroporous network shown in FIG. 1E in accordance with an embodiment of the invention.

FIG. 2 is a side view of a cross-section of a 3D printer 20 printing a breast implant 21 supported on a 3D printing stage 22. The 3D printer is shown with a first printhead 23, a second printhead 24 and a third printhead 25. The cross section of the implant 21 is shown with a load bearing macroporous network 26 with an open cell structure being filled with a first hydrogel 27 printed at the core of the implant, and a second hydrogel 28 printed to surround the first hydrogel. The first printhead is shown with a reservoir 29 containing pellets of a polymeric composition 32 for forming the load bearing macroporous network, the second printhead is shown with a reservoir 30 containing a pre-gel solution or slurry 33, and the third printhead is shown with a reservoir 31 containing a gelling agent 34.

FIG. 3 is a side view of a cross-section of a 3D printer 40 printing a breast implant 44 supported on a 3D printing stage 45. The 3D printer is shown with a first printhead 46, a second printhead 47, a third printhead 48 and a fourth printhead 49. The cross-section of the implant is shown with a load bearing network 41 with an open cell structure being filled with a first hydrogel 42 printed at the core of the implant, and a second hydrogel 43 printed to surround the first hydrogel. The first printhead 46 is shown with a reservoir 50 containing pellets of a polymeric composition 54 for forming the load bearing macroporous network, the second printhead 47 is shown with a reservoir 51 charged with a first pre-gel solution or slurry 55, the third printhead 48 is shown with a reservoir 52 charged with a second pre-gel solution or slurry 56, and the fourth printhead 49 is shown with a reservoir 53 charged with a gelling agent 57.

FIG. 4A is a side view of a cross-section of a beaker 60 containing a load bearing macroporous network 61 of a breast implant 62.

FIG. 4B is a side view of a cross section of a breaker 60 on a shaker 63. The beaker contains a load bearing macroporous network 61 of a breast implant 62 in a solution or slurry of a pre-gel 64. Arrows 65 show pre-gel 64 diffusing into the load bearing macroporous network.

FIG. 4C is a side view of a cross-section of a beaker 60 containing a load bearing macroporous network 61 of a breast implant 62. The breast implant is shown with a pre-gel 66 in the macroporous network being irradiated with light 67 to cross-link the pre-gel and form a hydrogel within the macroporous network.

FIG. 5A is a front view of an implant in accordance with another embodiment of the invention.

FIG. 5B is a cross sectional view of the implant shown in FIG. 5A taken along line A-A.

FIG. 5C is an isometric view of the implant shown in FIG. 5A.

FIG. 6A is a front view of an implant in accordance with another embodiment of the invention.

FIG. 6B is a cross sectional view of the implant shown in FIG. 6A taken along line B-B.

FIG. 6C is an isometric view of the implant shown in FIG. 6A.

7A is a front view of an implant having a cavity in accordance with another embodiment of the invention.

FIG. 7B is a cross sectional view of the implant shown in FIG. 7A taken along line A-A.

FIG. 7C is an isometric view of the implant shown in FIG. 7A.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

To further assist in understanding the following definitions are set forth below. However, it is also to be appreciated that unless defined otherwise as described herein, all technical and scientific terms used herein have the same meaning as commonly understood by one of ordinary skill in the art to which this invention belongs.

### I. Definitions

“Bioactive agent” as generally used herein refers to therapeutic, prophylactic or diagnostic agents, preferably agents that promote healing and the regeneration of host tissue, and also therapeutic agents that prevent, inhibit or eliminate infection. “Agent” includes a single such agent and is also intended to include a plurality.

“Biocompatible” as generally used herein means the biological response to the material or device being appropriate for the device's intended application in vivo. Any metabolites of these materials should also be biocompatible.

“Blend” as generally used herein means a physical combination of different polymers, as opposed to a copolymer formed of two or more different monomers.

“Compressive modulus” as used herein is measured with a universal testing machine at a cross-head speed of 20 mm min⁻¹. Samples are preloaded to engage the load and compressed at 5 to 15% strain. Clinically relevant cyclic load is repeated 10 times and compressive modulus is calculated based on secondary cyclic load due to the artifact caused by a take up of slack, and alignment or seating of the specimen. Compressive modulus may also be measured using ASTM standards ASTM D1621-16 or ASTM D695-15.

“Compression resilience” as used herein is calculated as the work done during compression recovery divided by the work done during compression multiplied by 100.

“Copolymers of poly-4-hydroxybutyrate” as generally used herein means any polymer containing 4-hydroxybutyrate with one or more different hydroxy acid units. The copolymers may be isotopically enriched.

“Copolymers of poly(butylene succinate)” as generally used herein means any polymer containing 1,4-butanediol and succinic acid units, and one or more different diol or diacid units. The copolymers may include one or more of the following: branching agent, cross-linking agent, chain extender agent, and reactive blending agent. The copolymers may be isotopically enriched.

“Degradable”, and any of its variants or derivatives including but not limited to “degrades”, “degraded” and “degradation”, as generally used herein means the material is degraded in the body, and the degradation products are eliminated or excreted from the body. The terms “absorbable”, “resorbable”, “degradable”, and “erodible”, and their respective variants or derivatives, with or without the prefix “bio”, can be used interchangeably herein, to describe materials broken down and gradually absorbed, excreted, or eliminated by the body, whether degradation is due mainly to hydrolysis or mediated by metabolic processes, and also includes processes where the material is dissolved or dispersed before absorption, excretion or elimination by the body.

“Endotoxin content” as generally used herein refers to the amount of endotoxin present in an implant or sample, and is determined by the limulus amebocyte lysate (LAL) assay.

“Infill density” as used herein is the ratio of volume occupied by the printed material in a porous implant divided by the total volume occupied by the printed material and the pore space expressed as a percentage.

“Molecular weight” as generally used herein, unless otherwise specified, refers to the weight average molecular weight (Mw), not the number average molecular weight (Mn), and is measured by GPC relative to polystyrene.

“Poly(butylene succinate) mean a polymer containing 1,4-butanediol units and succinic acid units. The polymer may include one or more of the following: branching agent, crosslinking agent, chain extender agent, and reactive blending agent. The polymer may be isotopically enriched.

“Poly(butylene succinate) and copolymers” includes polymers and copolymers prepared with one or more of the following: chain extenders, coupling agents, crosslinking agents and branching agents.

“Poly-4-hydroxybutyrate” as generally used herein means a homopolymer containing 4-hydroxybutyrate units. It can be referred to herein as P4HB or TephaFLEX® biomaterial (manufactured by Tepha, Inc., Lexington, MA). The polymers may be isotopically enriched.

“Subfascial” as used herein means under the connective tissue sheath (the outer fascia) of the pectoral muscle, but above the pectoral muscle.

“Soft tissue” as used herein means body tissue that is not hardened or calcified. Soft tissue excludes hard tissues such as bone and tooth enamel.

“Strength retention” refers to the amount of time that a material maintains a particular mechanical property following implantation into a human or animal. For example, if the tensile strength of a resorbable fiber or strut decreases by half over 3 months when implanted into an animal, the fiber or strut's strength retention at 3 months would be 50%.

“Sub-glandular” as used herein means under the breast tissue and above the pectoral muscle.

“Sub-pectoral” as used herein means at least partially under the pectoral muscle.

### II. Materials for Preparing Implants

In embodiments, the implants can be used to reshape the breast, fill voids in the breast, lift the breast, and augment the breast. The implants are soft tissue implants meaning that they can be used for soft tissue regeneration, augmentation, repair, reinforcement, and reconstruction. The implants can eliminate the need to use permanent breast implants during mastectomy, mastopexy and breast augmentation procedures. The implants are biocompatible, and are preferably replaced in vivo by the patient's tissue as the implants degrade. The implants are particularly suitable for augmentation of the breast, especially soft tissues of the breast. The implants preferably have a compressive modulus that allows the implant to temporarily deform under a compressive force, recover their shape from compression when the force is removed, and have a feel similar to breast tissue. Optionally, the implants can be coated or filled with autologous tissue, autologous fat, fat lipoaspirate, injectable fat, adipose cells, fibroblast cells, and stem cells prior to implantation, during implantation, or post-implantation.

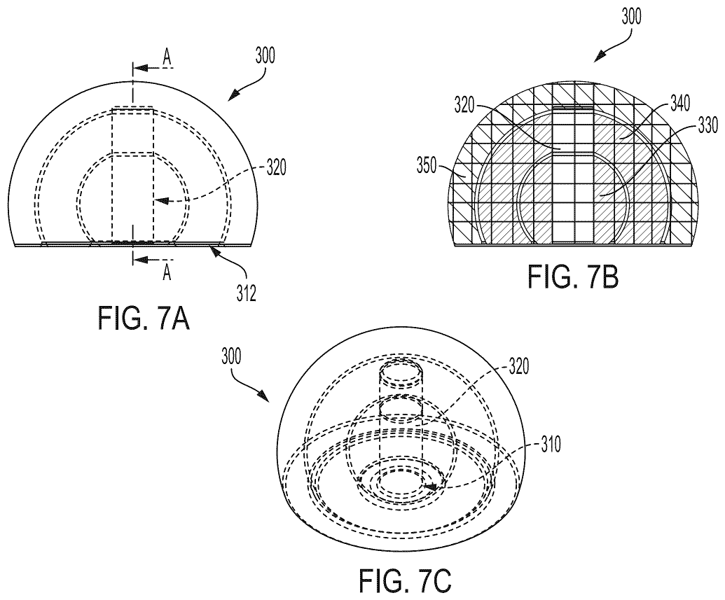

With reference to FIGS. 7A-7C, an implant 300 may further comprise one or more openings 310, including one or more passages or cavities 320, to allow insertion of a vascular pedicle or other tissue mass in the implant. In the embodiment shown in FIGS. 7A-7C, the cavity is cylindrical shaped and extends from an opening 310 in the back wall 312 towards the NAC of the breast. The macroporous network surrounding the open passageway carries the hydrogels, water-soluble polymers, or combinations thereof. As described herein, a plurality of different types of hydrogels and/or water-soluble polymers may be arranged in regional or zone layers (330, 340, 350) for timed absorption and to facilitate tissue ingrowth. It is to be understood that the cavity may have a wide variety of shapes and orientations and is only to be limited as recited in any appended claims. Additionally, although only one cavity is shown in FIGS. 7A-7C, implants may have a plurality of cavities. Preferably, the width or diameter of the cavity 320 ranges from 5 to 25 mm.

### A. Polymers and Hydrogels for Preparing Implants

In embodiments, the implants comprise a matrix with a load bearing macroporous network with an open cell structure, and are at least partly filled with one or more hydrogels, one or more water-soluble polymers, or a combination of one or more hydrogels and one or more water-soluble polymers. In embodiments, the implants comprise a first hydrogel or a first water-soluble polymer, and a second hydrogel or a second water-soluble polymer, where the second hydrogel degrades faster than the first hydrogel or the first water-soluble polymer and surrounds the first hydrogel or the first water-soluble polymer in the implant, or the second water-soluble polymer surrounds the first hydrogel or the first water-soluble polymer and the second water-soluble polymer degrades faster than the first hydrogel or the first water-soluble polymer in the implant. In embodiments, the implants further comprise a third hydrogel with a faster rate of degradation than the second hydrogel or the second water-soluble polymer, or a third water-soluble polymer with a faster rate of degradation than the second hydrogel or the second water-soluble polymer, and the third hydrogel or the third water-soluble polymer surrounds the second hydrogel or the second water-soluble polymer in the implant. In embodiments, the implants comprise one or more hydrogels and/or one or more water-soluble polymers with a faster degrading hydrogel or water-soluble polymer surrounding slower degrading hydrogels or water-soluble polymers. The load bearing macroporous networks may optionally comprise other features, such as one or more openings or passages, including one or more transverse passages.

The load bearing macroporous network of the implant may comprise permanent materials, such as non-degradable thermoplastic polymers, including polymers and copolymers of ethylene and propylene, including ultra-high molecular weight polyethylene, ultra-high molecular weight polypropylene, nylon, polyesters such as poly(ethylene terephthalate), poly(tetrafluoroethylene), polyurethanes, poly(ether-urethanes), poly(methylmethacrylate), polyether ether ketone, polyolefins, and poly(ethylene oxide). However, the load bearing macroporous network of the implant preferably comprises absorbable materials, more preferably thermoplastic or polymeric absorbable materials, and even more preferably the implant's load bearing macroporous network is made completely from absorbable materials.

In a preferred embodiment, the implant's load bearing macroporous network is made from one or more absorbable polymers or copolymers, preferably absorbable thermoplastic polymers and copolymers, and even more preferably absorbable thermoplastic polyesters. The implant's load bearing macroporous network may, for example, be prepared from polymers including, but not limited to, polymers comprising glycolic acid, glycolide, lactic acid, lactide, 1,4-dioxanone, trimethylene carbonate, 3-hydroxybutyric acid, 4-hydroxybutyrate, 3-hydroxyhexanoate, 3-hydroxyoctanoate, ε-caprolactone, including polyglycolic acid, polylactic acid, polydioxanone, polycaprolactone, copolymers of glycolic and lactic acids, such as VICRYL® polymer, MAXON® and MONOCRYL® polymers, and including poly(lactide-co-caprolactones); poly(orthoesters); polyanhydrides; poly(phosphazenes); polyhydroxyalkanoates; synthetically or biologically prepared polyesters; polycarbonates; tyrosine polycarbonates; polyamides (including synthetic and natural polyamides, polypeptides, and poly(amino acids)); polyesteramides; poly(alkylene alkylates); polyethers (such as polyethylene glycol, PEG, and polyethylene oxide, PEO); polyvinyl pyrrolidones (PVP); polyurethanes; polyetheresters; polyacetals; polycyanoacrylates; poly(oxyethylene)/poly(oxypropylene) copolymers; polyacetals, polyketals; polyphosphates; (phosphorous-containing) polymers; polyphosphoesters; polyalkylene oxalates; polyalkylene succinates; poly(maleic acids); silk (including recombinant silks and silk derivatives and analogs); chitin; chitosan; modified chitosan; biocompatible polysaccharides; hydrophilic or water soluble polymers, such as polyethylene glycol, or polyvinyl pyrrolidone (PVP), with blocks of other biocompatible or biodegradable polymers, for example, poly(lactide), poly(lactide-co-glycolide), or polycaprolactone and copolymers thereof, including random copolymers and block copolymers thereof.

Preferably the load bearing macroporous network of the implant is prepared from an absorbable polymer or copolymer that will be substantially resorbed after implantation within a 1 to 24-month timeframe, more preferably a 3 to 18-month timeframe, and retain some residual strength for at least 2 weeks to 6 months. In embodiments, the load bearing macroporous network has a strength retention of at least 50% at 3 months.

Blends of polymers and copolymers, preferably absorbable polymers, can also be used to prepare the implant's load bearing macroporous network. Particularly preferred blends of absorbable polymers are prepared from absorbable polymers including, but not limited to, polymers comprising glycolic acid, glycolide, lactic acid, lactide, 1,4-dioxanone, trimethylene carbonate, 3-hydroxybutyric acid, 4-hydroxybutyrate, C-caprolactone, 1,4-butanediol, 1,3-propane diol, ethylene glycol, glutaric acid, malonic acid, oxalic acid, succinic aid, adipic acid, or copolymers thereof.

In a particularly preferred embodiment, poly-4-hydroxybutyrate (Tepha's P4HB™ polymer, Lexington, MA) or a copolymer thereof is used to make the implant's load bearing macroporous network. Copolymers include P4HB with another hydroxy acid, such as 3-hydroxybutyrate, and P4HB with glycolic acid or lactic acid monomer. Poly-4-hydroxybutyrate is a strong, pliable thermoplastic polyester that is biocompatible and resorbable (Williams, et al. Poly-4-hydroxybutyrate (P4HB): a new generation of resorbable medical devices for tissue repair and regeneration, Biomed. Tech. 58(5):439-452 (2013)). Upon implantation, P4HB hydrolyzes to its monomer, and the monomer is metabolized via the Krebs cycle to carbon dioxide and water. In a preferred embodiment, the P4HB homopolymer and copolymers thereof have a weight average molecular weight, Mw, within the range of 50 kDa to 1,200 kDa (by GPC relative to polystyrene), more preferably from 100 kDa to 600 kDa, and even more preferably 200 kDa to 450 kDa. A weight average molecular weight of the polymer of 50 kDa or higher is preferred for processing and mechanical properties.

In another preferred embodiment, the load bearing macroporous network of the implant is prepared from a polymer comprising at least a diol and a diacid. In a particularly preferred embodiment, the polymer used to prepare the load bearing macroporous network is poly(butylene succinate) (PBS) wherein the diol is 1,4-butanediol and the diacid is succinic acid. The poly(butylene succinate) polymer may be a copolymer with other diols, other diacids or a combination thereof. For example, the polymer may be a poly(butylene succinate) copolymer that further comprises one or more of the following: 1,3-propanediol, ethylene glycol, 1,5-pentanediol, glutaric acid, adipic acid, terephthalic acid, malonic acid, methylsuccinic acid, dimethylsuccinic acid, and oxalic acid. Examples of preferred copolymers are: poly(butylene succinate-co-adipate), poly(butylene succinate-co-terephthalate), poly(butylene succinate-co-butylene methylsuccinate), poly(butylene succinate-co-butylene dimethylsuccinate), poly(butylene succinate-co-ethylene succinate) and poly(butylene succinate-co-propylene succinate). The poly(butylene succinate) polymer or copolymer may also further comprise one or more of the following: chain extender, coupling agent, cross-linking agent and branching agent. For example, poly(butylene succinate) or copolymer thereof may be branched or cross-linked by adding one or more of the following agents: malic acid, trimethylol propane, glycerol, trimesic acid, citric acid, glycerol propoxylate, and tartaric acid. Particularly preferred agents for branching or crosslinking the poly(butylene succinate) polymer or copolymer thereof are hydroxycarboxylic acid units. Preferably the hydroxycarboxylic acid unit has two carboxylic groups and one hydroxyl group, two hydroxyl groups and one carboxyl group, three carboxyl groups and one hydroxyl group, or two hydroxyl groups and two carboxyl groups. In one preferred embodiment, the implant's load bearing macroporous network is prepared from poly(butylene succinate) comprising malic acid as a branching or cross-linking agent. This polymer may be referred to as poly(butylene succinate) cross-linked with malic acid, succinic acid-1,4-butanediol-malic acid copolyester, or poly(1,4-butylene glycol-co-succinic acid), cross-linked with malic acid. It should be understood that references to malic acid and other cross-linking agents, coupling agents, branching agents and chain extenders include polymers prepared with these agents wherein the agent has undergone further reaction during processing. For example, the agent may undergo dehydration during polymerization. Thus, poly(butylene succinate)-malic acid copolymer refers to a copolymer prepared from succinic acid, 1,4-butanediol and malic acid. In another preferred embodiment, malic acid may be used as a branching or crosslinking agent to prepare a copolymer of poly(butylene succinate) with adipate, which may be referred to as poly[(butylene succinate)-co-adipate] crosslinked with malic acid. As used herein, “poly(butylene succinate) and copolymers” includes polymers and copolymers prepared with one or more of the following: chain extenders, coupling agents, crosslinking agents and branching agents. In a particularly preferred embodiment, the poly(butylene succinate) and copolymers thereof contain at least 70%, more preferably 80%, and even more preferably 90% by weight of succinic acid and 1,4-butanediol units. The polymers comprising diacid and diols, including poly(butylene succinate) and copolymers thereof and others described herein, preferably have a weight average molecular weight (Mw) of 10,000 to 400,000, more preferably 50,000 to 300,000 and even more preferably 100,000 to 200,000 based on gel permeation chromatography (GPC) relative to polystyrene standards. In a particularly preferred embodiment, the polymers and copolymers have a weight average molecular weight of 50,000 to 300,000, and more preferably 75,000 to 300,000. In one preferred embodiment, the poly(butylene succinate) or copolymer thereof used to make the load bearing macroporous network has one or more, or all of the following properties: density of 1.23-1.26 g/cm³, glass transition temperature of −31° C. to −35° C., melting point of 113° C. to 117° C., melt flow rate (MFR) at 190° C./2.16 kgf of 2 to 10 g/10 min, and tensile strength of 30 to 60 MPa.

In another embodiment, the polymers and copolymers described herein that are used to prepare the load bearing macroporous network of the implant, including P4HB and copolymers thereof and PBS and copolymers thereof, include polymers and copolymers in which known isotopes of hydrogen, carbon and/or oxygen are enriched. Hydrogen has three naturally occurring isotopes, which include ¹H (protium), ²H (deuterium) and ³H (tritium), the most common of which is the ¹H isotope. The isotopic content of the polymer or copolymer can be enriched for example, so that the polymer or copolymer contains a higher than natural ratio of a specific isotope or isotopes. The carbon and oxygen content of the polymer or copolymer can also be enriched to contain higher than natural ratios of isotopes of carbon and oxygen, including, but not limited to ¹³C, ¹⁴C, ¹⁷O or ¹⁸O. Other isotopes of carbon, hydrogen and oxygen are known to one of ordinary skill in the art. A preferred hydrogen isotope enriched in P4HB or copolymer thereof or PBS or copolymer thereof is deuterium, i.e. deuterated P4HB or copolymer thereof or deuterated PBS or copolymer thereof. The percent deuteration can be up to at least 1% and up to 5, 10, 15, 20, 25, 30, 35, 40, 45, 50, 55, 60, 65, 70, 75, 80 or 85% or greater.

In a preferred embodiment, the polymers and copolymers that are used to prepare the load bearing macroporous network, including P4HB and copolymers thereof and PBS and copolymers thereof, have low moisture contents. This is preferable to ensure the implants can be produced with high tensile strength, prolonged strength retention, and good shelf life. In a preferred embodiment, the polymers and copolymers that are used to prepare the implants have a moisture content of less than 1,000 ppm (0.1 wt %), less than 500 ppm (0.05 wt %), less than 300 ppm (0.03 wt %), more preferably less than 100 ppm (0.01 wt %), and even more preferably less than 50 ppm (0.005 wt %).

The compositions used to prepare the implants desirably have a low endotoxin content. In preferred embodiments, the endotoxin content is low enough so that the implants produced from the polymer compositions have an endotoxin content of less than 20 endotoxin units per device as determined by the limulus amebocyte lysate (LAL) assay. In one embodiment, the polymeric compositions used to prepare the load bearing macroporous network of the implant have an endotoxin content of <2.5 EU/g of polymer or copolymer. For example, the P4HB polymer or copolymer, or PBS polymer of copolymer have an endotoxin content of <2.5 EU/g of polymer or copolymer.

In embodiments, the implant's reinforced matrix comprising the macroporous network is filled with one or more hydrogels. Preferably, the hydrogels are degradable. Preferably, the hydrogels have different degradation rates when two or more hydrogels are present in the implant. In embodiments, the implants comprise a first hydrogel and a second hydrogel, and the second hydrogel degrades faster than the first hydrogel. In embodiments, the implants further comprise a third hydrogel, and the third hydrogel degrades faster than the second hydrogel.

In embodiments, the hydrogels are homopolymeric, copolymeric or multipolymer interpenetrating polymer hydrogels. The hydrogels may be amorphous, semicrystalline or crystalline. The hydrogels may comprise chemical or physical crosslinking. The hydrogels may be photo-crosslinked. The hydrogels may be nonionic, ionic, amphoteric electrolyte containing both acidic and basic groups or zwitterionic containing both anionic and cationic groups.

Examples of hydrogels that can be formed with physical crosslinking, for example, by heating a polymer solution, cooling a polymer solution, hydrogen bonding, or by ionic interaction include PEG-PLA, PEO-PPO, agarose, carrageenan, gelatin, Na+ alginate-+Ca2+, +2Cl−, Na+ alginate-polylysine, chitosan-polylysine, chitosan-TPP, and CMC. Examples of hydrogels that can be formed by chemical crosslinking include collagen-glutaraldehyde. Examples of hydrogels that can be formed by radiation crosslinking include PEG, and PVME.

In embodiments, an implant may comprise a first hydrogel and a second hydrogel with different degradation rates, and the first and second hydrogels may differ in the amount of crosslinking. The first hydrogel may be more crosslinked than the second hydrogel such that the first hydrogel degrades more slowly than the second hydrogel. In the embodiments, the implant may comprise a third hydrogel with a third different degradable rate, and a third different degree of crosslinking. The third hydrogel may be crosslinked such that the second hydrogel degrades more slowly than the third hydrogel. In embodiments, the hydrogels may differ only in the amount of crosslinking.

In embodiments, the hydrogels may be formed from pre-gels and gelling agents. For example, the gelling agents may be a crosslinking agent. The crosslinking agent may be added in different amounts or concentrations to pre-gels in order to produce hydrogels with different rates of degradation. The crosslinking agent may chemically or physically crosslink the hydrogel. The crosslinking agent may be a chemical agent, including organic and inorganic crosslinking agents, but it may also be a non-chemical agent, including for example light to photochemically crosslink a hydrogel. In the case of hydrogels that are formed by photo-crosslinking, hydrogels with different degradation rates may be formed from photo-crosslinkable pre-gels by using light of different intensity or for different durations. Photo-crosslinked hydrogels may also be formed with different degradation rates from a photo-crosslinkable pre-gel containing two or more different crosslinking chemistries that can be activated at different wavelengths of light.

In embodiments, the hydrogels and pre-gels may comprise natural polymers, including proteins, polypeptides, glycosaminoglycans, and polysaccharides. Examples include hydrogels and pre-gels comprising collagen, gelatin, fibrin, elastin, silk, starch, cellulose, methylcellulose, carboxymethylcellulose, hydroxypropyl methyl cellulose, hyaluronan, hyaluronic acid (HA), alginate, chitosan, carrageenan, pectin, pullulan, dextran, β-glucan, gellan, welan, xanthan, agarose, chondroitin sulfate, dermatan sulfate, heparin, keratin sulfate, albumin, casein, elastin, and resilin. The hydrogels and pre-gels may comprise synthetic polymers. Examples include hydrogels and pre-gels comprising polyvinyl alcohol, poly(vinyl methyl ether), polyethylene glycol, poly(ethylene glycol) diacrylate (PEGDA), poly(ethylene glycol) dimethacrylate (PEGDMA), polypropylene glycol, polyurethanes, polyphosphazenes, polypeptides, poly(N-isopropyl acrylamide), poly(vinylpyrrolidone), polymethacrylic acid, and polyacrylates and copolymers. In embodiments, the hydrogels and pre-gels may comprise both natural and synthetic components, for example, a methacrylated gelatin (GelMa) hydrogel, GelMa and PEGDA, alginate and polyacrylamide, and PVA and carrageenan. In embodiments, the hydrogels and pre-gels may comprise mixtures of natural polymers, for example, gelatin and alginate, gelatin and hyaluronic acid or derivative thereof, gelatin and agar.

In embodiments, the implant comprises a hyaluronic acid-based hydrogel (HA-based hydrogel). In embodiments, the HA-based hydrogel is formed by photo-crosslinking. In embodiments, the HA-based hydrogel is formed by thiol-ene photo-crosslinking. In embodiments, the HA-based hydrogel is formed by derivatization with norbornene, or derivative thereof such as norbornene carboxylic acid, and light initiated cross-linking of norbornene with a di-thiol crosslinker. Optionally, the norbornene derived HA-based hydrogel may be further derivatized with other ligands by light-initiated reaction with other mono-thiols or di-thiols. In embodiments, the implant comprises a norbornene modified alignate.

Additional pre-gels, gelling agents, crosslinkers, and hydrogels are set forth in Li et al. “3D PRINTING OF HYDROGELS: RATIONAL DESIGN STRATEGIES AND EMERGING BIOMEDICAL APPLICATIONS”, Mater. Sci. Eng, R 140 (2020) 100543, https://doi.org/10.1016/j.mser.2020.100543, as well as methods to print these 3D hydrogels.

In embodiments, the implant's reinforced matrix comprising the macroporous network is filled with one or more water-soluble polymers. Preferably, the water-soluble polymers are degradable over time from the implant. Preferably, the water-soluble polymers have different degradation rates when two or more water-soluble polymers are present in the implant. In embodiments, the implants comprise a first water-soluble polymer and a second water-soluble polymer, and the second water-soluble polymer degrades faster than the first water-soluble polymer. In embodiments, the implants further comprise a third water-soluble polymer, and the third water-soluble polymer degrades faster than the second water-soluble polymer.

Examples of water-soluble polymers that may be used to prepare the implants include: polyethylene glycol, polyvinyl pyrrolidone (PVP), polyvinyl alcohol (PVA), polyacrylic acids, polyacrylamides, polyphosphates, polyoxazolines, divinyl ether-maleic anhydride, N-(2-hydroxypropyl) methacrylamide, and polyphosphazenes.

### B. Additives

Certain additives may be incorporated into the implant. The additives may be incorporated in the matrix of the implant. The additives may be incorporated on the load bearing macroporous network of the implant, or in or on the one or more hydrogels, or one or more water-soluble polymers, of the implant including the surface of the implant. In one embodiment, one or more additives are incorporated with the polymers or copolymers described herein during a compounding process to produce pellets or fibers that can be subsequently processed to produce the load bearing macroporous network of the implant. For example, pellets or fibers may be 3D printed to form the load bearing macroporous network of the implant. In another embodiment, the pellets may be ground to produce powders suitable for further processing, for example, by 3D printing. Or, powders suitable for further processing, for example by 3D printing, may be formed directly by blending the additives and polymer or copolymer. If necessary, powders used for processing may be sieved to select an optimum particle size range. In another embodiment, the additives may be incorporated into the polymeric compositions used to prepare the load bearing macroporous network structure of the implants using a solution-based process. In embodiments, the one or more additives may be mixed with the one or more hydrogels, or one or more water-soluble polymers, and incorporated into the implant during filling of the implant with one or more hydrogels or one or more water-soluble polymers. In embodiments, the one or more additives may be coated on the one or more hydrogels, or one or more water-soluble polymers, after the implant has been filled with one or more hydrogels, or one or more water-soluble polymers. The one or more hydrogels, or one or more water-soluble polymers, may also be derivatized with one or more additives prior to incorporating the one or more hydrogels, or one or more water-soluble polymers, in the implant.

In a preferred embodiment, the additives are biocompatible, and even more preferably the additives are both biocompatible and absorbable.

In one embodiment, the additives may be nucleating agents and/or plasticizers. These additives may be added to the polymeric compositions used to prepare the load bearing macroporous network of the implant's matrix in sufficient quantity to produce the desired result. In general, these additives may be added in amounts between 1% and 20% by weight. Nucleating agents may be incorporated to increase the rate of crystallization of the polymer, copolymer or blend. Such agents may be used, for example, to facilitate fabrication of the load bearing macroporous network of the implant, and to improve the mechanical properties of the load bearing macroporous network of the implant. Preferred nucleating agents include, but are not limited to, salts of organic acids such as calcium citrate, polymers or oligomers of PHA polymers and copolymers, high melting polymers such as PGA, talc, micronized mica, calcium carbonate, ammonium chloride, and aromatic amino acids such as tyrosine and phenylalanine.

Plasticizers that may be incorporated into the polymeric compositions for preparing the load bearing macroporous network of the implant's matrix include, but are not limited to, di-n-butyl maleate, methyl laureate, dibutyl fumarate, di(2-ethylhexyl) (dioctyl) maleate, paraffin, dodecanol, olive oil, soybean oil, polytetramethylene glycols, methyl oleate, n-propyl oleate, tetrahydrofurfuryl oleate, epoxidized linseed oil, 2-ethyl hexyl epoxytallate, glycerol triacetate, methyl linoleate, dibutyl fumarate, methyl acetyl ricinoleate, acetyl tri(n-butyl) citrate, acetyl triethyl citrate, tri(n-butyl) citrate, triethyl citrate, bis(2-hydroxyethyl) dimerate, butyl ricinoleate, glyceryl tri-(acetyl ricinoleate), methyl ricinoleate, n-butyl acetyl rincinoleate, propylene glycol ricinoleate, diethyl succinate, diisobutyl adipate, dimethyl azelate, di(n-hexyl) azelate, tri-butyl phosphate, and mixtures thereof. Particularly preferred plasticizers are citrate esters.

### C. Bioactive Agents, Cells and Tissues

The implant's matrix can be loaded, filled, coated, or otherwise incorporated with bioactive agents. Bioactive agents may be included in the implant's matrix for a variety of reasons. For example, bioactive agents may be included in order to improve tissue ingrowth into the implant, to improve tissue maturation, to provide for the delivery of an active agent, to improve wettability of the implant, to prevent infection, and to improve cell attachment. The bioactive agents may also be incorporated into or onto the load bearing macroporous network structure of the implant's matrix, or into or onto the one or more hydrogels of the implant's matrix.

The implant's matrix can contain active agents designed to stimulate cell in-growth, including growth factors, cell adhesion factors including cell adhesion polypeptides, cellular differentiating factors, cellular recruiting factors, cell receptors, cell-binding factors, cell signaling molecules, such as cytokines, and molecules to promote cell migration, cell division, cell proliferation and extracellular matrix deposition. Such active agents include fibroblast growth factor (FGF), transforming growth factor (TGF), platelet derived growth factor (PDGF), epidermal growth factor (EGF), granulocyte-macrophage colony stimulation factor (GMCSF), vascular endothelial growth factor (VEGF), insulin-like growth factor (IGF), hepatocyte growth factor (HGF), interleukin-1-B (IL-1 B), interleukin-8 (IL-8), and nerve growth factor (NGF), and combinations thereof. As used herein, the term “cell adhesion polypeptides” refers to compounds having at least two amino acids per molecule that are capable of binding cells via cell surface molecules. The cell adhesion polypeptides include any of the proteins of the extracellular matrix which are known to play a role in cell adhesion, including fibronectin, vitronectin, laminin, elastin, fibrinogen, collagen types I, II, and V, as well as synthetic peptides with similar cell adhesion properties. The cell adhesion polypeptides also include peptides derived from any of the aforementioned proteins, including fragments or sequences containing the binding domains.

The implant's matrix can incorporate wetting agents designed to improve the wettability of the surfaces of the load bearing macroporous network, hydrogel or water-soluble polymer structures to allow fluids to be easily adsorbed onto the implant surfaces, and to promote cell attachment and or modify the water contact angle of the implant surface. Examples of wetting agents include polymers of ethylene oxide and propylene oxide, such as polyethylene oxide, polypropylene oxide, or copolymers of these, such as PLURONICS®. Other suitable wetting agents include surfactants, emulsifiers, and proteins such as gelatin.

Other bioactive agents that can be incorporated in the implant's matrix include antimicrobial agents, in particular antibiotics, disinfectants, oncological agents, anti-scarring agents, anti-inflammatory agents, anesthetics, small molecule drugs, anti-adhesion agents, inhibitors of cell proliferation, anti-angiogenic factors and pro-angiogenic factors, immunomodulatory agents, and blood clotting agents. The bioactive agents may be proteins such as collagen and antibodies, peptides, polysaccharides such as chitosan, alginate, hyaluronic acid and derivatives thereof, nucleic acid molecules, small molecular weight compounds such as steroids, inorganic materials such as hydroxyapatite and ceramics, or complex mixtures such as platelet rich plasma. Suitable antimicrobial agents include: bacitracin, biguanide, triclosan, gentamicin, minocycline, rifampin, vancomycin, cephalosporins, copper, zinc, silver, and gold. Nucleic acid molecules may include DNA, RNA, siRNA, miRNA, antisense or aptamers.

The implant's matrix may also contain allograft material and xenograft materials, including acellular dermal matrix material and small intestinal submucosa (SIS).

In embodiments, the implants may contain a vascular pedicle, vascular pedicle perforator, or other tissue mass. The vascular pedicle, vascular pedicle perforator, or other tissue mass may be autologous tissues, allograft tissues, or xenograft tissues.

In another embodiment, the implants may incorporate systems for the controlled release of the therapeutic or prophylactic agents.

In an embodiment, the implants are coated with autograft, allograft or xenograft tissue and cells prior to implantation, during implantation, or after implantation, or any combination thereof. In a particularly preferred embodiment, the implants are coated with autologous tissue and cells from the patient prior to implantation, during implantation, or after implantation, or any combination thereof. The autologous tissue and cells are preferably one or more of the following: autologous fat, fat lipoaspirate, fat tissue, injectable fat, adipose tissue, adipose cells, fibroblast cells, and stem cells, including human adipose tissue-derived stem cells, also known as preadipocytes or adipose tissue-derived precursor cells, and fibroblast-like stem cells. In one preferred embodiment, the implants may be coated with autologous tissue and cells as described herein, and may also further comprise a vascular pedicle, vascular pedicle perforator, or other tissue mass. As will be evident herein, the load bearing macroporous network structures of the implants are designed to create not only the shape of a breast implant, but also a large surface area that can retain autologous tissue and cells to encourage tissue in-growth.

### III. Methods for Preparing Multi-Component Breast Implants

A variety of methods can be used to manufacture the implants.

In embodiments, the implant is prepared so that it is able to provide one or more of the following: (i) structural support in the breast, (ii) a matrix with a load bearing macroporous network with an open structure for tissue ingrowth, (iii) an expanding volume for tissue ingrowth that increases over a period of time to permit tissue ingrowth and to prevent or minimize accumulation of fluid, (iv) controlled tissue ingrowth from the surface of the implant in the direction of the core of the implant, (v) a volume for tissue ingrowth that is capable of absorbing water and other biological fluids with high affinity, (vi) a slowly absorbing macroporous network engineered to allow complete tissue ingrowth of the implant prior to complete degradation, (vii) a macroporous network for delivering cells, tissue, fat, lipoaspirate, adipose cells, fibroblast cells, stem cells, and other bioactive agents to the breast, (viii) a structure that can allow incorporation of a graft into the implant structure, such as a vascular pedicle or other tissue mass, (ix) a structure that can be coated with cells, tissues, bioactive agents, including fat, lipoaspirate, adipose cells, fibroblast cells, and stem cells on the inside of the macroporous network by injection using a needle, (x) a load bearing macroporous network with an open cell structure that has a compressive strength of at least 0.1 kgf at 30% strain, (xi) a structure with a load bearing macroporous network with an open cell structure that has a compressive modulus of 0.1 kPa to 10 MPa at 5 to 15% strain, (xii) a structure with a load bearing macroporous network with an open cell structure that has a loss modulus of 0.3 to 100 kPa.

### A. Implant Shapes

In an embodiment, the implants are designed so that when manufactured, they are three-dimensional. In embodiments, the implants are designed to be used instead of permanent breast implants, such as silicone and saline breast implants.

The implant's shape allows the surgeon to increase tissue volume, reconstruct lost or missing tissue or tissue structures, contour tissues, augment tissues, restore tissue function, repair damaged tissue structures, enhance an existing tissue structure, increase soft tissue volume, alter the projection of the breast, increase upper pole fullness, and reshape the breast. In a preferred embodiment, the implants are used to reshape or repair the breast, augment the breast, and to repair the breast following mastectomy. In an embodiment, the implants allow the shape of soft tissue structures to be altered, or sculpted, without the use of permanent implants.

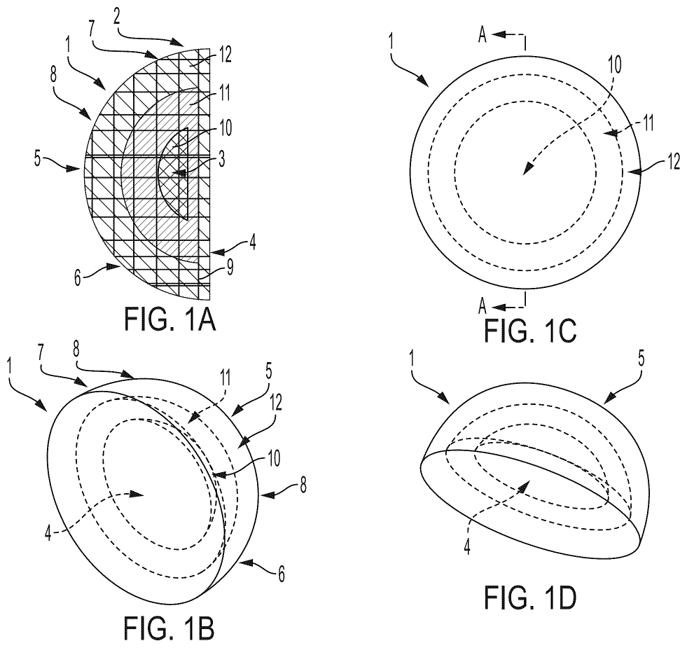

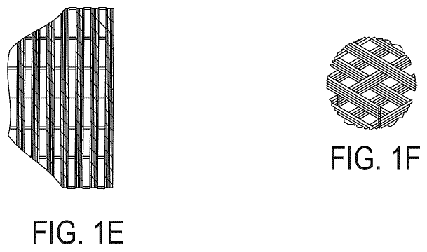

In embodiments, and with reference to FIG. 1A, a multi-component breast implant 1 comprising a reinforced matrix comprises a surface 2, a core 3, a back area 4 for placement on or near the chest wall of the patient, a front area 5 opposite the back area, a front bottom 6 for placement in the lower pole of the breast, a front top 7 for placement in the upper pole of the breast, a front intermediate-region 8 for placement under the skin of the patient, and a load bearing macroporous network 9 with an open cell structure. The multi-component breast implant 1 further comprises a first hydrogel 10, a second hydrogel 11, and a third hydrogel 12, with the third hydrogel present at the surface 2 of the implant. FIG. 1B shows an isometric view of a multicomponent breast implant 1 with a back area 4, a front area 5 opposite the back area, a front bottom 6, a front top 7, a front intermediate-region 8, and the locations within the implant of a first hydrogel 10, a second hydrogel 11, and a third hydrogel 12. FIG. 1C shows a top view of a cross section along the mid plane of a multicomponent breast implant 1, and the locations of the first hydrogel 10, the second hydrogel 11, and the third hydrogel 12. FIG. 1D shows an alternate isometric view of a multicomponent breast implant 1 with a back area 4 and a front area 5. In embodiments, one or more water-soluble polymers may be substituted for one or more of the hydrogels 10, 11 and 12.

The front area of the breast implant is shaped to provide projection to the breast. The projection of the implant as used herein is the maximum distance between the back area and the front area of the implant.

In embodiments, the front bottom area of the implant comprises a convex exterior surface. The convex exterior surface shape of the implant provides a pleasing anatomical shape to the lower pole of the breast.

Within the scope described herein, it should be understood that there are a plurality of implant shapes and dimensions, and that the invention is not limited with regard to the three-dimensional shape and dimensions of the implant, except where recited in the appended claims. The implants can be assembled or printed to have any size and shape suitable for use as an implant. For example, implants can easily be prepared that have three-dimensional shapes such as a: sphere, hemisphere, cylinder, teardrop, anatomical, cone, dome, cuboid, tetrahedron, triangular or square prism, dodecahedron, torus, and ellipsoid, and custom shapes can be produced optionally with the assistance of computer-aided design. For example, one can produce a dome shaped implant for the reconstruction of a breast.

The shape of the implant comprising the reinforced matrix is created by the load bearing macroporous network structure. For example, the dome shape of FIG. 1A is created by the load bearing macroporous network structure 9. The shape of the load bearing macroporous network may be changed to provide different implant shapes.

The implants may have different shapes in the front bottom and front top areas of the implant. The dimensions of the implant may be sized to augment breast tissue volume, to substitute for prior breast tissue volume, to change the volumetric distributions of breast tissue, to change the appearance of breast tissues, or to replace existing breast tissue volume with a smaller volume. The implants may be sized or shaped to provide a low, moderate, or high-profile shape to the breast, wherein the implant profile determines the projection of the breast. High profile shaped implants may be used to increase the height of the breast side wall, and provide patients with more upper pole fullness, or cleavage. Smaller increases in the height of the breast side wall may be obtained using implants with low or moderate profile shapes. The implants may be designed for use in the breast in sizes large enough to allow for their use in mastopexy and breast reconstruction. In embodiments, the breast implants have a volume between 100 and 1200 cc (cubic centimeters), and more preferably a volume between 120 and 850 cc. In embodiments, the implants are wide enough to span the width of a breast. In embodiments, the width of the back area of the implants is between 6 and 20 cm, and more preferably between 8 and 18 cm. The projection of the implant as used herein is the maximum distance between the back area and the front area of the implant. In embodiments, the projection of the implant is between 2 and 15 cm, more preferably between 3 and 10 cm, and even more preferably between 4 and 7 cm.

In a preferred embodiment, implants are provided in shapes that can be used to alter the soft tissue volume of a breast without the use of a permanent breast implant, such as a silicone breast implant. In embodiments, the implants can be prepared in shapes and sizes for use in augmenting the size of a breast, replacing the tissue volume and shape of the breast following a mastectomy procedure, to remove a defect in the breast, and to produce a specific appearance of the breast. For example, the implant can be prepared so that when implanted in the breast it produces a breast with a specific ratio of upper pole volume (UPV) to lower pole volume (LPV). In embodiments, the implant is a breast implant that has volumetric dimensions such that implantation of the implant results in a breast with an UPV of 25-35% of total breast volume, and LPV of 65-75% of total breast volume. In addition to sculpting the breast with specific volumetric ratios of tissue in the upper and lower poles, the dimensions and shape of the implant can also be chosen to provide very desirable shapes of the lower pole, upper pole, and extent of projection of the breast from the chest wall. In embodiments, the implants are designed so that (a) the lower pole of the breast has a very attractive lower pole curvature, specifically an attractive convex shape, (b) the upper pole of the breast has a straight or slightly concave curvature, and (c) the distance the breast projects from the breast wall is defined. It will therefore be apparent that the implants of the invention can be used to produce a very attractive reconstructed breast by having specific shapes that (i) define the ratio of the UPV to the LPV; (ii) define the curvatures of the upper and lower poles; (iii) define the extent of projection of the breast from the chest wall; and (iv) define the angulation of the nipple on the breast.

The shape of the implants may vary. Non-limiting examples of shapes include: round, teardrop, anatomically-breast shaped, or anatomically-breast contoured.

Additional shapes for the implant are set forth in U.S. patent application Ser. No. 16/262,018, filed Jan. 30, 2019 and entitled “FULL CONTOUR BREAST IMPLANT”, and incorporated herein by reference in its entirety.

In embodiments, the implants comprise one or more openings for insertion of one or more tissue masses. In a preferred embodiment, the implants comprise one or more openings on the back area of the implant (for example, back area 4 in FIG. 1A). One or more openings in the back area of the implant allow the surgeon to insert one or more pedicles into the implant when the back area of the implant is implanted on the chest wall. The one or more openings in the implant may create a chamber in the implant, or may create a passage through the implant. For example, an opening may extend from the back area 4 of the implant to the front area 5 of the implant. In embodiments, the implants may comprise an opening extending in a medial to lateral direction for insertion of a tissue mass. In embodiments, the implants may comprise one or more openings in the front area 5 of the implant, the front bottom 6, the front top 7 or the front intermediate-region 8. The dimensions of the one or more openings are sized to receive the tissue mass.

### B. Construction of the Implants

The implants comprise a reinforced matrix comprising a load bearing macroporous network with an open cell structure and one or more hydrogels, one or more water-soluble polymers, or a combination of hydrogels and water-soluble polymers.

In embodiments, the load bearing macroporous network may be prepared using textile processing, and may comprise filaments. For example, the macroporous network may be prepared by knitting, weaving or braiding of filaments. Filaments may be monofilaments, multifilaments and or yarns. In embodiments, the load bearing macroporous network is a 3D textile.

In embodiments, the macroporous network may be an orthogonal weave structure, multilayer structure, or an angle interlock weave structure. In embodiments, a 3D textile is formed by filament or yarn fed along two axes (x-axis and y-axis) plus an extra angular fed filament or yarn to create thickness. In embodiments, the macroporous network is a 3D knitted structure.

In embodiments, the load bearing macroporous network may be prepared using nonwoven textiles. In embodiments, the macroporous network comprises layers of nonwoven. In embodiments, the macroporous network may comprise a nonwoven 3D fabric. The nonwovens may comprise short filaments, including short yarn filaments. In embodiments, the nonwovens are melt blown, electrospun, derived from staple fibers, dry spun, prepared by centrifugal spinning, solution spun, spunlaid or spunbonded.

In embodiments, the macroporous network may comprise a 3D braided fabric. In embodiments, a 3D braided fabric may be formed by inter-plating three orthogonal sets of filaments or yarns.

In embodiments, the macroporous network may comprise a 3D composite. For example, the macroporous network may comprise a 3D woven composite, a 3D braided composite, a 3D stitched composite. In embodiments, a 3D composite is formed by applying a resin to a 3D preform such as a 3D woven composite, a 3D braided composite, or a 3D stitched composite.

In embodiments, the load bearing macroporous network is formed by particle leaching or phase separation. For example, a polymer solution comprising particles or porogens may be transferred into a suitable mold formed in the shape of the macroporous network, the solvent is then removed, for example, by evaporation or lyophilization, to leave the particles in the polymer structure. The mold may then be transferred to a bath to dissolve the particles or porogens forming the macroporous network.

In embodiments, the load bearing macroporous network is formed by foaming. In embodiments, the macroporous network is an open cell porous foam.

In embodiments, the load bearing macroporous network is formed by lamination. In embodiments, the laminate is perforated to form the macroporous network.

In embodiments, the load bearing macroporous network is formed from filaments or print lines by 3D printing. In one embodiment, the implant is prepared using 3D printing to construct the implant's load bearing macroporous network. 3D Printing of the macroporous network is highly desirable since it allows precise control of the shape of the implant's macroporous network. Suitable methods for 3D printing include extrusion-based additive manufacturing, fused deposition molding, fused filament fabrication, melt extrusion deposition, selective laser melting, printing of slurries and solutions using a coagulation bath, and printing using a binding solution and granules of powder. Preferably, the macroporous network of the implant is prepared by extrusion-based additive manufacturing, including fused deposition molding or fused filament fabrication.

In embodiments, the average diameters or width of the filaments or print lines of the load bearing macroporous network are 50 to 800 μm, more preferably 100 to 600 μm, and even more preferably 150 to 550 μm. In embodiments, the distances between the filaments of the implant are between 50 μm and 5 mm, more preferably 100 μm and 1 mm, and even more preferably 200 μm and 1 mm.

The average diameters of the filaments and the distances between the filaments may be selected according to the properties of the load bearing macroporous network that are desired, including the compression modulus and porosity. For example, the porosity of the macroporous network may be decreased by decreasing the infill density (defined as the ratio of volume occupied by filament material in the macroporous network divided by the total volume of the macroporous network expressed as a percentage) if the filament sizes, spacing between filaments, and print or textile pattern are kept constant. As the infill density decreases, the compression modulus also decreases if the filament sizes, spacing between filaments, and print pattern are kept constant. In embodiments, the infill density of the implant's macroporous network is from 1 to 60%, and more preferably from 5 to 25%.

In embodiments, the macroporous networks have pores with widths or diameters less than 15 mm, and preferably between 1 mm and 8 mm, and more preferably 2 mm to 5 mm. In embodiments, the pore sizes of the macroporous network of the implant are the same. In embodiments, the macroporous network of the implant comprises a mixture of pore sizes. Preferably, the load bearing macroporous network of the implant has an architecture that provides a larger surface area and large void volume suitable to allow the macroporous network to be colonized by cells and invaded by tissue, blood vessels, or combinations thereof, as the hydrogel or water-soluble polymer in the macroporous network is degraded.

In embodiments, the lateral porosity of the macroporous network can be less than or greater than the vertical porosity of the macroporous network.

In embodiments, filaments or print lines in the macroporous network are bonded to at least one other filament or print line.

In embodiments, the filaments or print lines of the macroporous network have surface roughness.

In embodiments, the properties of the macroporous network, such as compressive modulus, may also be changed by altering the 3D CAM (Computer Aided Design Model) for printing the loading bearing macroporous network.

In embodiments, the macroporous networks comprise unit cells. In embodiments, the networks may be prepared from unit cells with one or more of the following shapes: tetrahedron, cuboid, pentahedron, hexahedron, heptahedron, octahedron, icosahedron, decahedron, dodecahedron, tetradecahedron, and prisms, antiprisms, and truncated polyhedra thereof. Examples of unit cells in the shape of prisms, antiprisms and truncated polyhedra are a hexagonal prism, an octagonal antiprism, and a truncated dodecahedron. In an embodiment the unit cells are formed from elongated polyhedra. In a preferred embodiment, the unit cells have 4, 6, 8, 12 or 20 faces. In a particularly preferred embodiment, the unit cells are dodecahedrons, even more preferably rhombic dodecahedrons. In embodiments, the network may be made from two or more different unit cells, for example, a combination of dodecahedron and octagonal shapes. In embodiments, the sizes and shapes of the unit cells may be selected to provide different types of porous networks with different volumetric densities. The properties of the networks formed from the repeating unit cells are highly predictable, and can be predicted based on the dimensions of the unit cells, and the materials used to prepare the unit cells. Unit cells with different physical properties may be prepared by selection of the dimensions of the unit cells, geometries of the unit cells, and the material used to prepare the unit cells. Selecting specific unit cell dimensions and materials makes it possible to produce networks from the unit cells with properties that are compressible and soft. In embodiments, the mechanical properties of the network may be varied without changing the shape of the unit cells, but instead by varying the diameter or width of the filaments of the unit cells. For example, the required filament thickness or diameter of a given unit cell, made from a given polymer, can be calculated if a desired mechanical property is known, such as elastic modulus or compressive strength. In a preferred embodiment, the dimensions and materials of unit cells are selected such that implant networks prepared from the unit cells have properties that are similar to those of breast tissue. In a preferred embodiment, the unit cells of the macroporous networks can be compressed, and optionally recover their original shape when the compressive force is released.

In embodiments, the dimensions of the pores of the implant's macroporous network are large enough to allow needles to be inserted into the pores of the network in order to deliver bioactive agents, cells, fat, and other compositions by injection. In embodiments, the architecture of the macroporous network is designed to allow needles with gauges of 12-21 to be inserted into the network. This property allows the macroporous network to be loaded with cells, tissue, collagen, bioactive agents and additives, including fat, using a syringe and without damaging the network. In embodiments, the network allows insertion of needles into the network with outer diameters of 0.5 to 3 mm.

In embodiments, the implants comprise an external shell enclosing the reinforced matrix, or enclosing the load bearing macroporous network.

In embodiments, the load bearing macroporous network may be 3D printed to further comprise one or more openings for insertion of a tissue mass. The tissue mass is preferably a vascular pedicle. The opening is preferably formed on the back area 4 of the implant for insertion of a tissue mass. The opening may extend partly into the implant, or may extend from the back area 4 to the front area 5 of the implant. The implant may comprise an opening on the front bottom 6, front top 7 or front intermediate-region 8 of the implant. These openings may extend partly into the implant, or all the way through the implant.

In a typical procedure, the load bearing macroporous network of the implant is prepared by extrusion-based additive manufacturing, including melt extrusion deposition, fused pellet deposition, and fused filament fabrication, of a composition comprising an absorbable polymer or blend thereof.

The absorbable polymer or blend is preferably dried prior to printing to avoid a substantial loss of intrinsic viscosity. Preferably, the polymer or blend is dried so that the moisture content of the composition to be printed is no greater than 0.5 wt. % as measured gravimetrically, and more preferably no greater than 0.05 wt. %. The polymer or blend may be dried in vacuo. In a particularly preferred method, the polymer or blend is dried in a vacuum chamber under a vacuum of at least 10 mbar, more preferably of at least 0.8 mbar, to a moisture content of less than 0.03% by weight. Elevated temperatures below the melting point of the polymer may also be used in the drying process. Alternatively, the polymer may be dried by extraction into a solvent and re-precipitation of the polymer, or with the use of desiccants. The moisture content of the polymer or blend may be determined using a VaporPro Moisture Analyzer from Arizona Instruments, or similar instrument.

In an embodiment, the load bearing macroporous network of the implant is formed by extrusion-based additive manufacturing, including fused pellet deposition, fused filament fabrication, and melt extrusion deposition of poly-4-hydroxybutyrate (P4HB). P4HB polymer (Mw of 100-600 kDa) is preferably dried as described above for 3D printing. The polymer may be pelletized and optionally ground for fused pellet deposition modeling and melt extrusion deposition, or extruded into suitable filaments for 3D printing by fused filament fabrication.

P4HB pellets or filament may be 3D printed by melt extrusion-based additive manufacturing to form the macroporous network of a breast implant using, for example, the printing parameters shown in Table 1, an Arburg Freeformed 200-3X 3D printer, and a 3D CAD (Computer Aided Design) Model for the implant's load bearing macroporous network. The average diameters of the 3D filaments that are printed from the P4HB polymer are selected based upon the properties of the load bearing macroporous network desired, including the network's compression modulus, and porosity or infill density. Preferably, the average filament diameters or widths are 50 to 800 μm, more preferably 100 to 600 μm, and even more preferably 150 to 550 μm.

TABLE 1
Parameters for melt extrusion-based additive
manufacturing of P4HB Macroporous network

| | |
|---|---|
| Print head temp (° C.) | 185 |
| Barrel zone 2 (° C.) | 135 |
| Barrel zone 1 (° C.) | 100 |
| Build chamber temp (° C.) | 10-15° C. |
| Screw speed (m/min) | 4 |
| Back pressure (Bar) | 50 |
| Recovery stroke (mm) | 6 |
| Deco speed (mm/s) | 2 |
| Deco stroke (mm) | 4 |
| Discharge nr (%): | 40-80 |
| In Filling density (%) | 5-60 |
| Drop ratio | 1-1.3 |

In another embodiment, the parameters shown in Table 2 may be used for melt extrusion-based additive manufacturing of the load bearing macroporous network using a composition comprising poly(butylene succinate) or copolymer thereof, and an Arburg Freeformed 200-3X 3D printer.

TABLE 2
Parameters for melt extrusion-based additive
manufacturing of PBS Macroporous network

| | |
|---|---|
| Print head temp (° C.) | 185-200 |
| Barrel zone 2 (° C.) | 135-150 |
| Barrel zone 1 (° C.) | 110 |
| Build chamber temp (° C.) | 10-80 |
| Screw speed (m/min) | 4 |
| Back pressure (Bar) | 50 |
| Recovery stroke (mm) | 6 |
| Deco speed (mm/s) | 2-4 |
| Deco stroke (mm) | 4-8 |
| Discharge nr (%): | 120 |
| In Filling density (%) | 30-100 |
| Drop ratio | 1-1.6 |

In embodiments, the implant comprises a reinforced matrix comprising a load bearing macroporous network at least partly filled by one or more hydrogels, one or more water-soluble polymer, or a combination thereof.

In embodiments, the load bearing macroporous network may be filled with a hydrogel and/or water-soluble polymer by 3D printing. In one embodiment, the load bearing macroporous network is at least partially filled with a hydrogel by 3D printing a pre-gel solution or slurry from one printhead, and adding a gelling agent to the pre-gel solution from a second printhead. Addition of the gelling agent to the pre-gel solution results in formation of the hydrogel in the load bearing macroporous network. In embodiments, the hydrogel may be formed from a pre-gel solution or slurry by printing the pre-gel solution, and irradiating the pre-gel to form a hydrogel rather than adding a gelling agent. This method may be used, for example, when the pre-gel solution or slurry can be crosslinked by irradiation with light (i.e. the method may be used in the preparation of photo-crosslinkable hydrogels).

The concentrations of the pre-gel solutions or slurries used in 3D printing the implants is dependent upon the molecular weight of the pre-gel. In embodiments, pre-gel solutions or slurries are printed from solutions or slurries of a pre-gel with a concentration of 1-20 wt %, more preferably 1-10 wt %, and even more preferably 2-5 wt %. For example, an alginate pre-gel may be printed at a concentration of 2-5 wt %, and a solution of calcium chloride used as a gelling agent. In embodiments, the pre-gel solutions may comprise a mixture of pre-gels. For example, a pre-gel solution may comprise 3% alginate and 9% methylcellulose, and be gelled with a solution of calcium chloride.

In embodiments, two hydrogels with different degradation rates may be 3D printed to at least partially fill the implant from the same pre-gel solution or slurry, by increasing or decreasing the crosslinking of the pre-gel solution or slurry. For example, a first hydrogel may be printed in the implant from a pre-gel solution or slurry and crosslinked with a gelling solution, and a second hydrogel may be printed from the same pre-gel solution but crosslinked less with the same gelling solution by using a lower concentration of the gelling solution or reducing the time of exposure of the pre-gel to the gelling solution (e.g. reducing the drops of gelling solution applied by the printer). Alternatively, for example, a first hydrogel may be printed in the implant from a pre-gel solution or slurry and crosslinked by irradiation with light, and a second hydrogel printed in the implant from the same pre-gel solution but crosslinked less by decreasing the time of exposure of the pre-gel to the light or decreasing the intensity of the light.

In embodiments, two hydrogels may be printed in the implant by printing two different pre-gel solutions or slurries. A first hydrogel may be printed in the macroporous network of the implant by printing a first pre-gel solution or slurry in the macroporous network, and a first gelling agent. A second hydrogel may be printed in the macroporous network of the implant by printing a second pre-gel solution or slurry in the macroporous network, and a gelling agent. Depending upon the choice of the second hydrogel, the gelling agent may be the same as the first gelling agent or different. In embodiments, the first pre-gel and first gelling agent are printed to fill the core of the macroporous network with a first hydrogel, and a second pre-gel and gelling agent are printed to form a second hydrogel surrounding the first hydrogel.

In embodiments, the load bearing macroporous network is at least partially filled with a water-soluble polymer by 3D printing a filament or resin of a water-soluble polymer. For example, a filament of polyvinyl alcohol may be used to at least partially fill a macroporous network. In embodiments, a polyvinyl alcohol filament may be 3D printed at an extrusion temperature of 205-220° C.

Preferably, the implants are prepared using a 3D printer setup with multiple printheads wherein the printer can print the macroporous network of the implant and the one or more hydrogels and/or one or more water-soluble polymers of the implant in a single manufacturing step.

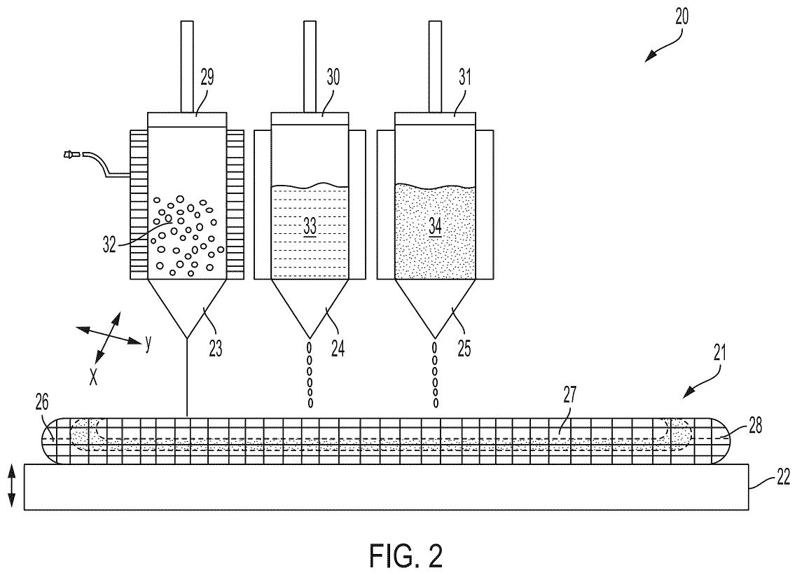

A suitable setup 20 for 3D printing an implant comprising a reinforced matrix comprising a load bearing macroporous network and two hydrogels is shown in FIG. 2. In this embodiment, the 3D printer comprises 3 printheads 23, 24, and 25, and a 3D printing stage 22. The reservoir 29 of the first printhead 23 is charged with pellets 32 of a polymeric composition for printing the load bearing macroporous network. The reservoir 30 of the second printhead 24 is charged with a pre-gel solution or slurry 33, and the reservoir 31 of the third printhead 25 is charged with a gelling agent 34. FIG. 2 shows the implant 21 being printed on the printing stage 22 by simultaneously printing the loading bearing macroporous network 26 with an open cell structure, and filling it with a first hydrogel 27 and a second hydrogel 28 surrounding the first hydrogel 27. The printing of the macroporous network and filling of the network with the first and second hydrogels is performed according to a 3D CAD Model for the implant. The first hydrogel 27 may be formed in the macroporous network by printing a solution or slurry of a pre-gel 33 from the second print head 24, and adding a first concentration or amount of a gelling agent 34 from the third printhead 25. The second hydrogel 28 may be formed in the macroporous network so that it surrounds the first hydrogel 27 by printing a solution or slurry of a pre-gel 33 from the second printhead 24, and adding a second concentration or amount of gelling agent 34 from the third printhead. The first and second concentrations or amounts of gelling agent 34 may be selected to form the first and second hydrogels wherein the second hydrogel will degrade faster than the first hydrogel. For example, a higher concentration or amount of the gelling agent 34 is added to the pre-gel 33 to form the first hydrogel 27 than is added to form the second hydrogel 28. In embodiments, printhead 23 may be designed to print filament instead of pellets.

In another embodiment, a similar equipment setup to that shown in FIG. 2 may be used to 3D print an implant comprising a reinforced matrix comprising a macroporous network and two hydrogels wherein the first and second hydrogels are formed by photo-crosslinking instead of using a gelling agent. In this embodiment, the third printhead 25 may be replaced with a suitable light source for photo-crosslinking the pre-gel 33. In this embodiment, hydrogels with different rates of degradation may be formed by irradiating the pre-gel for different times or with light of different intensity or with light of different wavelengths. Irradiating the pre-gel for a longer period of time may, for example, be used to form a more highly crosslinked and slower degrading hydrogel than is formed when the pre-gel is irradiated for a shorter period of time.

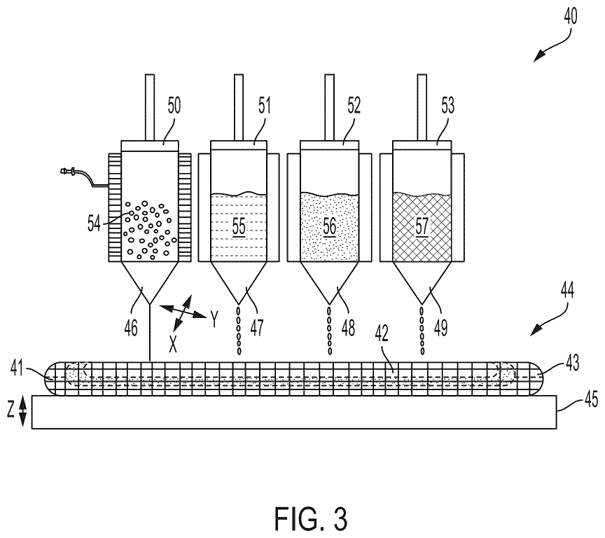

In embodiments, a suitable setup 40 for 3D printing both the load bearing macroporous network 41 and two hydrogels 42 and 43 of the implant using two different pre-gels is shown in FIG. 3. In this embodiment, the 3D printer comprises 4 printheads 46, 47, 48, and 49 and a 3D printing stage 45. The reservoir 50 of the first printhead 46 is charged with pellets 54 of a polymeric composition for printing the load bearing macroporous network. The reservoir 51 of the second printhead 47 is charged with a first pre-gel solution or slurry 55. The reservoir 52 of the third printhead 48 is charged with a second pre-gel solution or slurry 56. And the reservoir 53 of the fourth printhead 49 is charged with a gelling agent 57. FIG. 3 shows the implant 44 being printed on the printing stage 45 by simultaneously printing the loading bearing macroporous network 41 with an open cell structure, and filling it with a first hydrogel 42 and a second hydrogel 43 surrounding the first hydrogel 42. The printing of the macroporous network and filling of the network with the first and second hydrogels is performed according to a 3D CAD Model for the implant. The first hydrogel 42 may be formed in the macroporous network by printing a solution or slurry of the first pre-gel 55 from the second print head 47, and adding a gelling agent 57 from the fourth printhead 49. The second hydrogel 43 may be formed in the macroporous network so that it surrounds the first hydrogel 42 by printing a solution or slurry of a second pre-gel 56 from the third printhead 48, and adding a gelling agent 57 from the fourth printhead. The first and second hydrogels may be selected and formed so that the second hydrogel degrades faster than the first hydrogel. In embodiments, printhead 46 may be configured to print filament instead of pellets 54.

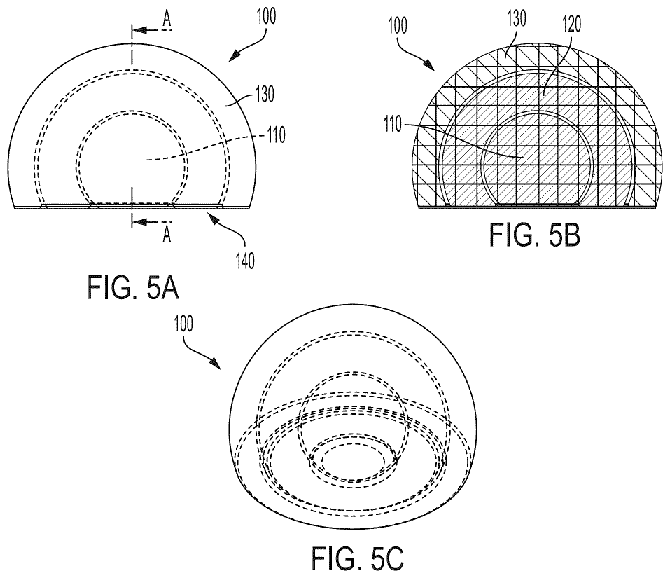

In embodiments, and with reference to FIGS. 5A-5C, the implants may be printed so that all three hydrogels are in contact with the chest wall. In this embodiment, each hydrogel comes into contact with the chest wall when the implant is implanted. In embodiments, the implants may be printed so that a second hydrogel 120 surrounds a first hydrogel 110, except in the back area 140 of the implant 100 where the hydrogels will be in contact with the chest wall. In embodiments, one or two water-soluble polymers may be substituted for one or both of the hydrogels. For example, an implant may be prepared by 3D printing a water-soluble polymer, such as polyvinyl alcohol, instead of a second hydrogel 120, to surround a first hydrogel 110. Polyvinyl alcohol may be 3D printed, for example, from polyvinyl alcohol filament at an extrusion temperature of 205-220° C. In embodiments, the implants may be printed wherein the implants comprise a third hydrogel 130 surrounding the second hydrogel 120 and a second hydrogel surrounding the first hydrogel 110, except at the back 140 of the implant 100. In embodiments, one, two or three water-soluble polymers may be substituted for one or more of the hydrogels 110, 120 and 130.

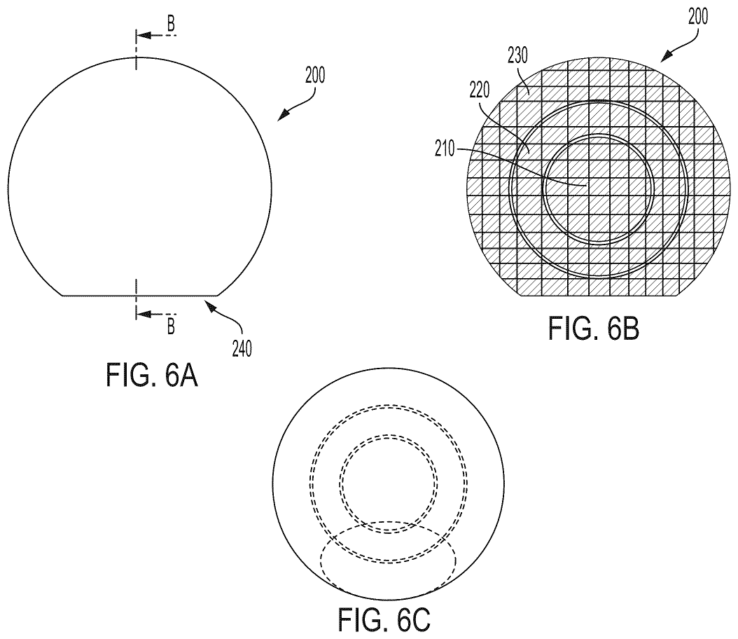

In embodiments, and with reference to FIGS. 6A-6C, all hydrogels (210, 220, 230) of the implant 200 are present at the back area 240 of the implant. In embodiments, one or more water-soluble polymers may be substituted for one or more of the hydrogels.

In embodiments, all hydrogels (210, 220, 230) of the implants, or one or more water-soluble polymers of the implants, can come into contact with the chest wall when the implant is implanted in a patient.

In embodiments, the implants may be filled at least partly with hydrogels and/or water-soluble polymers without the use of 3D printing.

In embodiments, the implants are prepared by immersing a load bearing macroporous network with an open cell structure in a solution or slurry of a pre-gel, allowing the pre-gel to penetrate the macroporous network, removing the solution or slurry of the pre-gel, and crosslinking the pre-gel. In embodiments, the pre-gel is photo-crosslinked by exposure to light to form the hydrogel within the macroporous network.

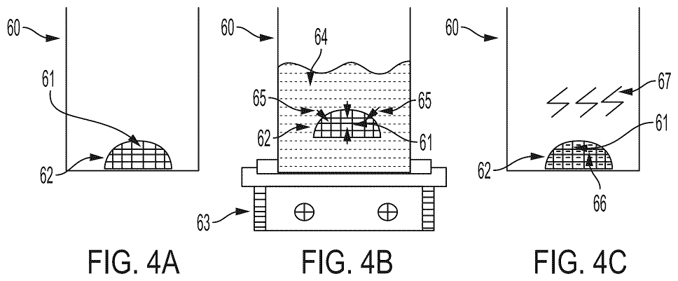

FIGS. 4A-C illustrate in an embodiment how an implant may be prepared by first allowing a pre-gel to penetrate a macroporous network, and then crosslinking the pre-gel to provide a macroporous network at least partly filled with a hydrogel. FIG. 4A shows a side view of a cross-section of a beaker 60 containing a load bearing macroporous network 61 of an implant 62. The load bearing macroporous network may be prepared, for example, by 3D Printing as described herein. As shown in FIG. 4B, the beaker may be filled with a solution or slurry of a pre-gel 64 that immerses or covers the macroporous network 61. The beaker is placed on a shaker 63, and shaken to facilitate diffusion of the pre-gel into the macroporous network as illustrated by the direction of the arrows 65 in FIG. 4B. Once the pre-gel has diffused into the macroporous network, the residual solution or slurry of pre-gel is removed from the beaker. FIG. 4C shows the macroporous network 61 filled with a pre-gel 66 after the network has been immersed in the solution or slurry of pre-gel, and the solution or slurry removed. The pre-gel 66 retained in the macroporous network 61 may then be crosslinked to form the hydrogel in the macroporous network. In the example shown in FIG. 4C, the pre-gel is crosslinked by exposure of the pre-gel 66 to light 67. In an alternative embodiment, the pre-gel 66 may be crosslinked by adding a gelling agent to the pre-gel. The method shown in FIGS. 4A-C is most suited to the preparation of an implant comprising a macroporous network filled with one hydrogel, and preferably to the preparation of implants with photo-crosslinkable hydrogels. In embodiments, the photo-crosslinkable pre-gels include norbornene derivatized hyaluronic acid-based pre-gels. These pre-gels can be crosslinked, for example, by exposure to UV light with a wavelength of 320-390 nm in the presence of a di-thiol crosslinker.

### C. Properties of the Implant

In an embodiment, the mechanical properties of the implant are designed so that the mechanical properties of the implant approximate the mechanical properties of breast tissue. In embodiments, the mechanical properties of the implant are designed so that the mechanical properties of the reinforced matrix comprising the load bearing macroporous network of the implant approximate the mechanical properties of breast tissue.

In embodiments, the compressive modulus of the implant or macroporous network of the implant at 5 to 15% strain is 0.1 kPa to 10 MPa, more preferably 0.3 kPa to 1 MPa, and even more preferably 3 kPa to 200 kPa. In embodiments, the compressive modulus of the implant or macroporous network allows the implant to be compressed when a compressive force is applied, but recover from compression when the compressive force is removed.

In another embodiment, the implant or macroporous network of the implant has a compressive modulus at 5 to 15% strain that is ±50% of the compressive modulus of breast tissue. In other embodiments, the implant or macroporous network has a compressive modulus at 5 to 15% strain that is ±50%, more preferably ±25% of the compressive modulus of glandular tissue, adipose tissue, skin, pectoralis fascia, or breast tissue.

In embodiments, the filaments present in the macroporous network of the breast implant are formed from a polymeric composition. The polymeric composition preferably has one or more of the following properties: (i) an elongation at break greater than 100%; (ii) an elongation at break greater than 200%; (iii) a melting temperature of 60° C. or higher, (iv) a melting temperature higher than 100° C., (v) a glass transition temperature of less than 0° C., (vi) a glass transition temperature between −55° C. and 0° C., (vii) a tensile modulus less than 300 MPa, and (viii) a tensile strength higher than 25 MPa.

In embodiments, the filaments present in the macroporous network of the breast implant have one or more of the following properties: (i) breaking load of 0.1 to 200 N, 1 to 100 N, or 2 to 50 N; (ii) elongation at break of 10% to 1,000%, more preferably 25% to 500%, and even more preferably greater than 100% or 200%, and (iii) elastic modulus of 0.05 to 1,000 MPa, and more preferably 0.1 to 200 MPa.

In embodiments, the macroporous network of the implant may have anisotropic properties. That is, the macroporous network may have different properties in different directions. For example, the macroporous network may have a first compressive modulus in one direction, and a second different compressive modulus in a second direction. In embodiments, the macroporous networks of the breast implants may have different properties in the direction from the font top to the front bottom of the implant versus the properties of the implant measured from a lateral to medial direction when implanted in the breast.

In order to allow tissue in-growth into the macroporous network of the implant, the macroporous network should have a strength retention long enough to permit cells and blood vessels to invade the implant's macroporous network and proliferate. In embodiments, the macroporous network of the implant has a strength retention of at least 25% at 2 weeks, more preferably at least 50% at 2 weeks, and even more preferably at least 50% at 4 weeks. In other embodiments, the macroporous network of the implant is designed to support mechanical forces acting on the implant, and to allow a steady transition of mechanical forces from the macroporous network to regenerated host tissues. In particular, the macroporous network of the implant is designed to support mechanical forces acting on the implant, and to allow a steady transition of mechanical forces from the macroporous network to new host tissues.

### D. Other Features of the Implants

The implants, reinforced matrix, or macroporous networks of the implants may be trimmed or cut with scissors, blades, other sharp cutting instruments, or thermal knives in order to provide the desired implant or macroporous network shapes. The implants or macroporous networks can also be cut into the desired shapes using laser-cutting techniques. This can be particularly advantageous in shaping filament-based implants because the technique is versatile, and importantly can provide shaped implants and macroporous networks without sharp edges.

The implant may comprise suture tabs so that the implants can be anchored in the body using for example sutures and or staples. The number of tabs may vary. In one embodiment, the number of tabs will depend upon the load exerted on the implant. A larger number of tabs may be desirable when the implant is heavier or has a larger volume. In embodiments, the implant comprises 1, 2, 3, 4, 5, 6, 7, 8, 9, 10, 11, 12, 13, 14, 15, 16, 17, 18, 19, 20 tabs or more. In embodiments, the implant preferably contains 4 or more tabs, preferably 4-12 tabs, in order to anchor the breast implant to the chest wall. The dimensions of the tabs are preferably from 0.5 cm×0.5 cm to 5 cm×4 cm, and more preferably 2 cm×2.5 cm. The tabs attached to the implant must have sufficient strength retention in vivo to resist mechanical loads, and to allow sufficient in-growth of tissue into the implant in order to prevent subsequent movement of the implant after implantation. In a preferred embodiment, the suture pullout strength of the tabs attached to the implant, is greater than 10 N, and more preferably greater than 20 N.

### E. Implant Coatings and Fillings

The implant comprises a macroporous network wherein there is a continuous path through the implant which encourages and allows tissue ingrowth into the network structure as the hydrogel(s) and/or water-soluble polymer(s) degrade. The continuous path also allows the entire macroporous network structure to be coated with one or more of the following: cells, stem cells, fat cells, adipose cells, tissues, collagen, additives, and bioactive agents.

Macroporous networks with low infill densities, for example, less than 60%, or 5-25%, are preferred because they provide a large void space for tissue ingrowth.

In embodiments, the implants are fabricated with coatings and or some or all of the reinforced matrix or macroporous network is used as a carrier. For example, the macroporous network may be fabricated by populating some or all of the void space of the macroporous network with one or more of the following: cells and tissue, including autograft, allograft or xenograft tissue and cells, and vascularized pedicle. Examples of cells that can be inserted into the void spaces of the implant's macroporous network, and coated on the surfaces of the network, include adipose cells, fibroblast cells, and stem cells. In embodiments, a vascularized pedicle may be inserted into void space of the implant's macroporous network. In embodiments, the implants may be coated partially or fully with one or more bioactive agents. Particularly preferred bioactive agents that can be coated on the implant's macroporous network include collagen and hyaluronic acid or derivative thereof. In other embodiments, the implant or the implant's macroporous network may be coated with one or more antibiotics.

### IV. Methods for Implanting the Implants

In embodiments, the implant is implanted into the body. Preferably, the implant is implanted into a site of reconstruction, remodeling, repair, and or regeneration. In a preferred embodiment, the implant is implanted in the breast of a patient. In a preferred embodiment, connective tissue and or vasculature will invade the macroporous network of the implant after implantation, and as the hydrogel(s) and/or water-soluble polymer(s) degrade. In a particularly preferred embodiment, the implant comprises absorbable materials, and connective tissue and or vasculature will also invade the spaces where the hydrogels and/or water-soluble polymers have degraded (and eventually where the macroporous network has absorbed). The pores of the macroporous network may be colonized by cells prior to implantation or, more preferably, following implantation, and the pores of the implant's macroporous network invaded by tissue, blood vessels or a combination thereof.

The implant's macroporous network may be coated or filled with transplantation cells, stem cells, fibroblast cells, adipose cells, and or tissues prior to implantation, or after implantation. In embodiments, the implant's macroporous network is coated with differentiated cells prior to, or subsequent to, implantation. Differentiated cells have a specific form and function. An example is a fat cell. In embodiments, the implant's macroporous network is populated with cells by injection, before or after implantation, and preferably by using needles that do not damage the macroporous network of the implant. The implant's macroporous network may also be coated or filled with platelets, extracellular adipose matrix proteins, gels, hydrogels, water-soluble polymers, and bioactive agents prior to implantation. In an embodiment, the implant's macroporous network may be coated with antibiotic prior to implantation, for example, by dipping the implant in a solution of antibiotic.

The implants may be used to deliver autologous cells and tissue to the patient in the breast. The autologous tissue is preferably one or more of the following: autologous fat, fat lipoaspirate, injectable fat, adipose cells, fibroblast cells, and stem cells.

The implants may be used to deliver fat tissue into a patient. In a particularly preferred embodiment, autologous fatty tissue is prepared prior to, or following, implantation of the implant, and is injected or otherwise inserted into or coated on the implant's macroporous network prior to or following implantation of the implant. The autologous fatty tissue is preferably prepared by liposuction at a donor site on the patient's body. After centrifugation, the lipid phase containing adipocytes is then separated from blood elements, and combined with the implant's macroporous network prior to implantation, or injected, or otherwise inserted into the implant's macroporous network following implantation. In an embodiment, the implant's macroporous network is injected with, or filled with, a volume of lipoaspirate that represents 1% to 50% of the total volume of the macroporous network, and more preferably 1% to 20% of the total volume of the macroporous network.

In another embodiment, lipoaspirate fatty tissue taken from the patient may be mixed with a biological or synthetic matrix, such as very small fibers or particles, prior to adding the lipoaspirate to the implant's macroporous network. In this embodiment, the added matrix serves to hold or bind micro-globules of fat, and disperse and retain them within the macroporous network of the implant. In some embodiments, the use of added matrix can help to prevent pooling of fat which could lead to necrosis, and or help to increase vascularization of the implant.

In another embodiment, a vascular pedicle or other tissue mass is harvested from the patient, and inserted into the implant. The pedicle or other tissue mass may be inserted into the implant prior to implantation of the implant, and then the implant with the pedicle or other tissue mass implanted in the patient, or the pedicle or other tissue mass may be inserted into the implant after the implant has been implanted in the patient.

In an embodiment, an implant is implanted and fixated in both breasts. In embodiments, the implants are implanted in patients during mastopexy and augmentation procedures, including revision procedures. In a particularly preferred embodiment, the implant is implanted in a patient that has undergone a: (i) mastectomy, (ii) breast lift and has need of augmentation, (iii) breast reduction and needs support, lift or remodeling of the reduced breast, or (iv) previous silicone or saline breast implant surgery and desires the silicone or saline implant to be removed and that a subsequent reconstruction of the breast will provide a fuller or large sized breast. The implant may also be implanted in a breast surgery patient to increase the projection of the breast away from the chest, and optionally additional fat graft volume added to the implant after implantation to increase the projection. Additional fat graft volume may be added to the implant immediately after implantation of the implant, but may also be added at follow up visits. For example, additional fat graft volume may be added to the implant on one or more occasions that are days, weeks, or months following the implantation of the implant. The procedures described herein can also be performed with removal of breast tissue, resection and redistribution of breast tissue.

In an embodiment, a method of implantation of the implant in the breast comprises at least the steps of: (i) making at least one incision to gain access to the breast tissue of the patient, (ii) separating the skin and subcutaneous fascia from the breast mound of the breast, (iii) positioning the implant on the breast mound of the breast, (iv) securing the implant to the tissue surrounding the breast mound of the breast, and (v) closing the incisions in the breast. This method may further comprise one or more of the following steps: (a) preparing a sample of lipoaspirate, and coating or filling the implant with the sample prior to implantation of the implant, (b) preparing a sample of lipoaspirate, and coating or filling the implant with the sample after implantation of the implant, preferably by injecting the sample into the implant, (c) inserting a vascular pedicle into the implant prior to, or after, implantation of the implant, and (d) suturing or stapling the implant in place. In a preferred embodiment, the implant is implanted in a sub-glandular, sub-pectoral or pre-pectoral position. In embodiments, the implant is sutured to the tissue surrounding the breast mound, and even more preferably to the fascia surrounding the pectoral muscle underlying the breast mound. In another embodiment, the implant comprises tabs, and the tabs are sutured to the tissue surrounding the breast mound.

The implant's reinforced matrix or macroporous network may also be coated or filled with cells and tissues other than fat grafts prior to, or subsequent to, implantation, as well as with cytokines, platelets and extracellular adipose matrix proteins. For example, the implant's reinforced matrix or macroporous network may be coated or filled with cartilage or dermal grafts. The implant's macroporous network may also be coated or filled with other tissue cells, such as stem cells genetically altered to contain genes for treatment of patient illnesses.

In an embodiment, the implant has properties that allow it to be delivered by minimally invasive means through a small incision. The implant may, for example, be designed so that it can be rolled, folded or compressed to allow delivery through a small incision. This minimally invasive approach can reduce patient morbidity, scarring and the chance of infection. In embodiments, the implant has a three-dimensional shape and shape memory properties that allow it to assume its original three-dimensional shape unaided after it has been delivered through an incision and into an appropriately sized dissected tissue plane. For example, the implant may be temporarily deformed by rolling it up into a small diameter cylindrical shape, delivered using an inserter, and then allowed to resume its original three-dimensional shape unaided in vivo. In embodiments, the implant is compressible and may be delivered into the breast through a funnel, such as a Keller funnel. In embodiments, the implant may be delivered into the breast through a funnel with a neck diameter (the narrowest part of the funnel) of 1 to 3 inches, and more preferably 1.5 to 2.5 inches. In embodiments, the implant may be delivered through a funnel with a neck diameter of 1 to 3 inches, and recover at least 70% of the implant's volume after delivery through the neck of the funnel into the breast. In embodiments, the implant has a compressive modulus to allow delivery of the implant through a funnel with a neck diameter of 1 to 3 inches, and the implant is able to recover at least 70% of the implant's volume after passage through the funnel's neck.

1. A breast implant comprising a reinforced matrix, wherein the implant comprises a surface, a core, a back area for placement on the chest wall of a patient, a front area opposite the back area, the front area comprising a front bottom for placement in the lower pole of a breast, a front top for placement in the upper pole of the breast, and a front intermediate-region for placement under skin of the patient, and wherein the reinforced matrix comprises a load bearing macroporous network with an open cell structure at least partly filled with one or more degradable hydrogels, degradable water-soluble polymers, or combinations thereof.

2. The implant of claim 1, wherein the reinforced matrix comprises a first hydrogel and a second hydrogel, wherein the second hydrogel surrounds the first hydrogel, or the reinforced matrix comprises a first water-soluble polymer and a second water-soluble polymer, wherein the second water-soluble polymer surrounds the first water-soluble polymer, or the reinforced matrix comprises a first hydrogel and a second water-soluble polymer, wherein the first hydrogel surrounds the second water-soluble polymer or the second water-soluble polymer surrounds the first hydrogel.

3. The implant of claim 2 wherein the reinforced matrix comprises a first hydrogel and a second hydrogel and wherein the second hydrogel degrades in vivo faster than the first hydrogel, or wherein the reinforced matrix comprises a first water-soluble polymer and a second water-soluble polymer and wherein the second water-soluble polymer degrades in vivo faster than the first water-soluble polymer, or wherein the reinforce matrix comprises a first hydrogel and a second water-soluble polymer and wherein one of the first hydrogel and second water-soluble polymer degrades faster than the other of the first hydrogel and the second water-soluble polymer.

4. The implant of claim 3 wherein the reinforced matrix comprises a first hydrogel and a second hydrogel, wherein the implant further comprises a third hydrogel, and wherein the second hydrogel is surrounded by the third hydrogel, and wherein the third hydrogel degrades in vivo faster than the second hydrogel.

5. The implant of claim 1, wherein the load bearing macroporous network has one or more of the following properties: a compressive strength of at least 0.1 kgf at 30% strain, a compressive modulus of 0.1 kPa to 10 MPa at 5 to 15% strain, and a loss modulus of 0.3 to 100 kPa.

6. The implant of claim 1, wherein the implant has a teardrop shape, anatomical shape, round shape, dome-like shape, or wherein the front bottom of the implant has a convex exterior surface.

7. The implant of claim 1, wherein the implant further comprises an opening for insertion of tissue into the implant.

8. The implant of claim 1, wherein the implant further comprises an external shell enclosing the reinforced matrix.

9. The implant of claim 1, wherein the implant further comprises one or more anchors, fasteners or tabs to fixate the implant in the breast.

10. The implant of claim 1, wherein the load bearing macroporous network is degradable or comprises one or more absorbable polymers.

11. The implant of claim 10, wherein the one or more absorbable polymers comprises, or is prepared from, one or more monomers selected from the group: glycolide, lactide, glycolic acid, lactic acid, 1,4-dioxanone, trimethylene carbonate, 3-hydroxybutyric acid, 3-hydroxybutyrate, 3-hydroxyhexanoate, 3-hydroxyoctanoate, 4-hydroxybutyric acid, 4-hydroxybutyrate, ε-caprolactone, 1,4-butanediol, 1,3-propane diol, ethylene glycol, glutaric acid, malic acid, malonic acid, oxalic acid, succinic aid, and adipic acid, or wherein the polymeric composition comprises poly-4-hydroxybutyrate or copolymer thereof, or poly(butylene succinate) or copolymer thereof.

12. The implant of claim 1, wherein the load bearing macroporous network comprises filaments, fibers, struts, textile, nonwoven, foam, laminate, or a phase separated or particle leached structure.

13. The implant of claim 12, wherein the filaments, fibers or struts have one of the following properties: a tensile strength higher than 25 MPa, a tensile modulus less than 300 MPa, an elongation at break greater than 100%, a melting temperature of 60° C. or higher, a glass transition temperature of less than 0° C., an average diameter or width of 10 μm to 5 mm, a breaking load of 0.1 to 200 N, and an elastic modulus of 0.05 to 1,000 MPa.

14. The implant of claim 12, wherein the load bearing microporous network is absorbable.

15. The implant of claim 1, wherein one or more of the degradable hydrogels or one or more of the water-soluble polymers degrades faster than the load bearing macroporous network with an open cell structure.

16. The implant of claim 1, wherein the load bearing macroporous network with an open cell structure comprises unit cells, and wherein the unit cells are selected from one or more of the following shapes: (i) tetrahedron, cuboid, pentahedron, hexahedron, heptahedron, octahedron, icosahedron, decahedron, dodecahedron, tetradecahedron, and prisms, antiprisms, and truncated polyhedra thereof; (ii) elongated polyhedra, (iii) shapes with 4, 6, 8, 12 or 20 faces; and (iv) rhombic dodecahedron.

17. The implant of claim 1, wherein the implant further comprises autologous fat, fat lipoaspirate, injectable fat, adipose cells, fibroblast cells, stem cells, hyaluronic acid, collagen, an antimicrobial agent, an antibiotic, a bioactive agent, and a diagnostic device.

18. A method of manufacturing the breast implant of claim 1, wherein the load bearing macroporous network with an open cell structure is manufactured by forming a macroporous network of filaments by 3D printing a polymeric composition.

19. The method of claim 18, wherein the macroporous network comprises unit cells, and wherein the unit cells are selected from one or more of the following shapes: (i) tetrahedron, cuboid, pentahedron, hexahedron, heptahedron, octahedron, icosahedron, decahedron, dodecahedron, tetradecahedron, and prisms, antiprisms, and truncated polyhedra thereof; (ii) elongated polyhedra, (iii) shapes with 4, 6, 8, 12 or 20 faces; and (iv) rhombic dodecahedron.

20. The method of claim 18, wherein the method further comprises loading at least one of a first hydrogel or a first water-soluble polymer in the macroporous network.

21. The method of claim 20, wherein the method further comprises loading at least one of a second hydrogel or a second water-soluble polymer in the macroporous network so that the second hydrogel or the second water-soluble polymer surrounds the first hydrogel or the first water-soluble polymer, and optionally, loading at least one of a third hydrogel or a third water-soluble polymer in the macroporous network so that the third hydrogel or the third water-soluble polymer surrounds the second hydrogel or second water-soluble polymer.

22. The method of claim 18, wherein the macroporous network of filaments is formed by extrusion-based additive manufacturing, selective laser melting, fused deposition modeling, fused filament fabrication, melt extrusion deposition, printing of a polymer slurry or solution using a coagulation bath, and printing using a binding solution and granules of polymer powder.

23. The method of claim 18, wherein the implant is formed using a 3D printer with two or more print heads, wherein the load bearing macroporous network with an open cell structure is formed by using a first print head to print the network from a polymeric composition, and a second print head is used to print at least one of a first hydrogel or a first water-soluble polymer so that it is located in the macroporous network.

24. The method of claim 23, wherein the 3D printer comprises a third print head, and the third print head is used to print at least one of a second hydrogel or a second water-soluble polymer in the macroporous network so that it surrounds the first hydrogel or the first water-soluble polymer.

25. The method of claim 23, wherein the implant is formed using solution-based or slurry-based printing to print the first and second hydrogels or first and second water-soluble polymers.

26. The method of claim 18, wherein the macroporous network has one or more of the following properties: a compressive strength of at least 0.1 kgf at 30% strain, a compressive modulus of 0.1 kPa to 10 MPa at 5 to 15% strain, and a loss modulus of 0.3 to 100 kPa.

27. A method of implanting an implant as recited in claim 1 in a breast comprising: (i) making at least one incision to gain access to breast tissue of the patient, (ii) separating skin and subcutaneous fascia from the breast mound of the breast, (iii) positioning the implant sub-glandular, sub-pectoral, or subfascial, (iv) securing the implant to nearby tissue, and (v) closing the incisions in the breast.

28. The method of claim 27, further comprising coating or injecting into the implant one or more of the following on one or more occasions either prior to implanting the implant in the patient or after implanting the implant in the patient: autologous fat, fat lipoaspirate, injectable fat, adipose cells, fibroblast cells, stem cells, gel, hydrogel, hyaluronic acid or derivative thereof, collagen, antimicrobial, antibiotic, and a bioactive agent.

29. The method of claim 27, further comprising inserting a vascular pedicle or other tissue mass in the implant.

30. (canceled)

31. (canceled)

32. A breast implant comprising a reinforced macroporous network defining a plurality of interconnected voids; and a first set of sacrificial void occupiers adapted to temporarily occupy a first set of voids until tissue grows therein, wherein the first set of sacrificial void occupiers is formed of at least one of a hydrogel, a water-soluble polymer, or a combination thereof.

33. The breast implant of claim 32, further comprising a second set of voids arranged to surround the first set of voids, and a second set of sacrificial void occupiers adapted to temporarily occupy the second set of voids until tissue grows therein, and wherein the second set of void occupiers is absorbable prior to the first set of void occupiers.

34. The breast implant of claim 33, wherein the first set of sacrificial void occupiers is a first hydrogel.

35. The breast implant of claim 34, wherein the first and second set of sacrificial void occupiers, and macroporous network are fabricated and arranged together to form the implant by 3D printing.

36. The breast implant of claim 34, wherein the second set of sacrificial void occupiers is a second hydrogel.

37. The breast implant of claim 36, wherein the second set of sacrificial void occupiers is absorbed before the first set of void occupiers, and the macroporous network completely degrades after the first set of void occupiers is degraded.

38. The breast implant of claim 37, wherein first set of void occupiers is absorbed within one year of implantation in the breast.

39. A method of implanting an implant as recited in claim 1 in a breast comprising delivering the implant through a funnel into the breast.

40. A breast implant comprising a reinforced matrix, wherein the implant comprises a surface, a core, a back area for placement on the chest wall of a patient, a front area opposite the back area, the front area comprising a front bottom for placement in the lower pole of a breast, a front top for placement in the upper pole of the breast, and a front intermediate-region for placement under skin of the patient, wherein the reinforced matrix comprises a load bearing macroporous network with an open cell structure, wherein the reinforced matrix comprises at least a first hydrogel or a first water-soluble polymer, and at least a second hydrogel or a second water-soluble polymer, wherein the second hydrogel or second water-soluble polymer surrounds the first hydrogel or the water-soluble polymer except in the back area of the implant.

41. The breast implant of claim 40 wherein the first hydrogel or first water-soluble polymer includes a first hydrogel and the second hydrogel or water-soluble polymer includes a second water-soluble polymer.
